(19) United States
(12) Patent Application Publication
(10) Pub. No.: US 2025/0354873 A1
(43) Pub. Date: Nov. 20, 2025

(54) TEMPERATURE INTEGRITY SENSOR

(71) Applicant: UNIVERSITÀ DEGLI STUDI DI CAGLIARI, Cagliari (IT)

(72) Inventor: Giuseppe SFORAZZINI, Cagliari (IT)

(73) Assignee: UNIVERSITÀ DEGLI STUDI DI CAGLIARI, Cagliari (IT)

(21) Appl. No.: 18/871,843

(22) PCT Filed: Jun. 20, 2023
(86) PCT No.: PCT/IB2023/056359

§ 371 Date: Dec. 5, 2024

(30) Foreign Application Priority Data
Jun. 20, 2022 (IT) 102022000013021

(51) Int. Cl. G01K 1/024; C07D 498/18; G01K 11/16

(52) U.S. Cl. CPC G01K 1/024; C07D 498/18; G01K 11/16

(57) ABSTRACT

A temperature integrity sensor or more precisely a temperature continuity sensor of a product which needs to be kept at a temperature below its degradation temperature, is provided. The product includes a refrigerated or frozen edible product; a pharmaceutical product such as a vaccine, or an antibiotic; or a biological-medical product such as a sample of a body fluid or tissue, or an organ. The sensor is based on RFID technology, in particular passive RFID technology.

### CROSS REFERENCE TO THE RELATED APPLICATIONS

This application is the national phase entry of International Application No. PCT/IB2023/056359, filed on Jun. 20, 2023, which is based upon and claims priority to Italian Patent Application No. 102022000013021, filed on Jun. 20, 2022, the entire contents of which are incorporated herein by reference.

### TECHNICAL FIELD

The present invention relates to a temperature integrity sensor or more precisely a temperature continuity sensor of a product which needs to be kept at a temperature below its degradation temperature, such as for instance a refrigerated, or frozen edible product; a pharmaceutical product such as a vaccine, or an antibiotic; or a biological-medical product such as a sample of a body fluid or tissue, or an organ.

The sensor is based on RFID technology, in particular passive RFID technology.

### BACKGROUND

Temperature control in the cold chain (Cold Supply Chain, CSC) is crucial to ensure the quality, safety, wholesomeness, and shelf life of perishable products. Medical products and foods are two main areas where CSC could cause harm to the public if not properly controlled. Every year, pharmaceutical companies develop drugs and vaccines that require cold storage to maintain their effectiveness. At the same time, perishable food products must be stored at temperatures below their “danger zone” to reduce the growth or spread of bacteria throughout the production and distribution chain, starting with producers and ending with consumers, who are often many kilometers away from each other.

Current methods of monitoring temperature rely primarily on the use of data loggers and radio frequency identification (RFID) tags (FIG. 1A). However, these devices measure the temperature only at certain time intervals or when they are interrogated by a reader (FIG. 1B), thus leaving no trace of any temperature variations that could occur during the interval between one measurement and another. More explicitly, these sensors fail to monitor any changes in the cold chain, so a product exposed to temperatures outside the optimal ones for its preservation and subsequently brought back to them, would easily evade controls.

Failure to monitor the persistence of low temperatures could therefore lead to serious problems affecting the integrity of sensitive products, e.g. vaccines. Thus the development and implementation of a safe, reliable, easily usable, low temperature persistence control system would represent a huge and distinct technological advance.

Nowadays, the tracking/monitoring of individual commercial items is generally entrusted to the application of radio frequency identification labels on the product packaging or on the product itself.

The latter are wireless devices with electronic circuitry made up of two basic components, an antenna to receive/analyse signals and a memory microchip (tag) to memorize an identification code. These devices communicate with an RFID reader to infer the identity of the object to which the tag is attached.

Tags can be “active” or “passive”. The former contain an on-board battery to power the internal circuit and generate radio waves. The seconds have no battery, and are powered directly by radio waves transmitted by the RFID reader which resonates with the antenna. RFIDs, both active and passive, designed for their use as temperature sensors integrate, in addition to their traditional circuitry, a component for detecting the temperature in real time. The information acquired by this additional component is then transferred to the reader as a variation of the initial radio signal (FIG. 1B).

Passive RFID tags have attracted considerable attention on the development of organic-based wireless sensors. The tag's simple architecture allows for low-cost, high-sensitivity, and easy-to-use sensors for monitoring food spoilage, toxic substances, and biomolecule screening. These devices are commonly prepared by contacting a thin film of electron-rich (p-type) or electron-poor (n-type) organic semiconductors (π-conjugated organic compounds) with the metal circuitry of a passive RFID. The organic layer at the heterojunction (contact surface between the thin film and the metal of the RFID) acts as a signal “modulator” of the device, varying some of the intrinsic physical properties of the RFID circuit, e.g. frequency, impedance (Z) and reflection coefficient (S11). Any variation to the π system of the organic semiconductor leads to a change in the physical properties of the RFID device which can be recorded as a variation of its radio frequencies.

The structural variations of the π system of an organic semiconductor determine the modulation of its conductivity. These changes are usually obtained in two ways: by means of chemical reactions with a specific analyte (chemical species, biological entities, etc.), and by means of changes in the geometric conformation of the π-system. As mentioned above, the first method is commonly used for preparing chemical sensors, which can be successfully interfaced to RFID devices for wireless reading of analyses. These sensors are based on the chemical transformations of the π system caused by the reaction of the analyte with the molecules or polymers that make up the organic layer. The second method has been mainly used for the preparation of colorimetric (non-RFID) thermal sensors which exploit the changes in the optical activity (e.g. absorption spectrum, emission spectrum) of organic semiconductors in a liquid medium.

The latter sensors exploit the variations of the geometry of the π conjugation, induced by the different rotational freedom of the molecules at different temperatures. Here, the high temperature favors the rotation along the sigma molecular bonds of the π-conjugated system, allowing the formation of distorted geometries, wherein the aromatic sub-units of the π-conjugated system do not rest on the same conjugation plane. The sigma molecular bond wherein two aromatic sub-units are out of the conjugation plane (relative to each other) is a “node”. The node is a point where electron delocalization (conjugation) is interrupted or significantly reduced. Thus, in twisted π-conjugated systems, the effective conjugation of the molecule is bounded by the planar molecular segments contained between two nodes, and not by the total number of aromatic sub-units of the conjugated molecule. Thus, conjugated systems subjected to high temperatures commonly assume conjugation-limited conformations, and consequently have localized optical activity around the UV spectrum. On the contrary, low temperatures suppress the rotation along the sigma molecular bonds of the π-conjugated system, favoring the planarization of the π-conjugated system, with consequent delocalization of the electrons along the whole conjugation, which results in an optical activity centered in the visible spectral range. In this type of sensors, the variation between the optical activity results in consequent color variations.

As already indicated above, to the best of our knowledge, RFID devices do not have the ability to measure temperature and store the information except at the price of complex circuitry, e.g. battery, thermocouples, memories, which would make the same devices bulkier, heavier, more expensive, and less suitable for their use as monitoring “labels” for single units, even and above all if the products to be monitored/controlled are small.

At the same time, the organic semiconductors used in RFID devices as an alternative to complex circuitry, can only measure the changes induced by chemical reactions in real time, thus mainly enabling the development of chemical and biological sensors.

To date, therefore, there are no:
passive RFID devices (therefore with reduced circuitry) capable of measuring the temperature or its variation and storing the information;
organic semiconductors that can be used as thermal sensors of cold in RFID devices, capable of measuring temperature variations and storing their chronological trace.

Semiconductor organic compounds based on the architecture called “Photochromic Torsional Switch” (PTS), which allows said π-conjugated organic compounds to regulate their optical and electronic properties by means of light stimuli, are known and described in patent U.S. Pat. No. 10,144,744. These materials are designed to undergo a conformation change of their π-conjugated system via the photo-isomerization of a “molecular actuator” inserted along the conjugation extension (FIG. 2). With this approach, the materials will be less conductive in their initial conformation (twisted/interrupted π-conjugated system) and more conductive after their exposure to light (planarized π-conjugated system). The first conformation (twisted/poorly conductive conjugation) can also be obtained spontaneously starting from the second conformation (planarized/conductive π conjugate system) by means of thermal relaxation, since the geometry assumed by the PTS molecular actuator in the first conformation is energetically more stable than that assumed in the second conformation. This thermal relaxation process is slowed down (or blocked) at temperatures around 0° C. and accelerated at temperatures above ambient (about 20° C.). In the patent it is mentioned that organic compounds based on the PTS architecture can be used as semiconductors in the active layer of organic optoelectronic devices, such as organic light emitting diodes (OLEDs), organic solar cells (OSCs), and organic field effect transistors (OFETs). However, the possibility of using the thermal relaxation of PTS compounds to monitor a temperature change has never been discussed.

As a solution to the above technological limitations and problems, the present invention proposes to use a new class of organic semiconductors (described in U.S. Pat. No. 10,144,744) as active material to be interfaced with passive RFID devices.

Unless specifically excluded in the detailed description that follows, what is described in this chapter is to be considered as an integral part of the detailed description of the invention.

### SUMMARY

It is highly desirable to have available a device that implements a precise, convenient and low-cost method for assessing the temperature integrity status of perishable products in order to avoid their deterioration when their optimum storage temperature is not maintained.

It is therefore an object of the present invention to develop an electronic device which is or which comprises a temperature integrity sensor based on RFID technology, in particular on passive RFID technology, which comprises as active material to be interfaced with said RFID, one or several compounds selected from those described in the general formula of U.S. Pat. No. 10,144,744.

Another object of the invention is to develop a temperature integrity sensor based on RFID technology, in particular on passive RFID technology, comprising a substrate and at least one layer on said substrate, which comprises at least one of the compounds identified above.

Another object of the invention is to develop a temperature integrity sensor based on RFID technology, in particular on passive RFID technology, comprising an oligomer or a polymer comprising one or more PTS units and one or more residues selected from those described in the general formula of U.S. Pat. No. 10,144,744.

Another object of the invention is to develop a composition comprising one or more compounds selected from those described in the general formula of U.S. Pat. No. 10,144,744 and a carrier such as, toluene, tetrahydrofuran, chloroform, chlorobenzene, 2-methyl-tetrafuran, 1,2,4-trimethylbenzene, 1,2,4-trichlorobenzene, o-xylene, anisole, 1,2-dichlorobenzene, methanol, water, isopropyl alcohol, acetone, ethyl acetate, cyclopentyl methyl ether; and/or an additive, such as 1,4-diiodobutane, 1,6-diiodohexane, hexadecane, 4-bromoanisole, nitrobenzene, 1-methyl-2-pyrrolidone, diphenyl ether, cyclopentyl methyl ether, 1-methylnaphthalene, 1-chloronaphthalene, diethylene glycol dibutyl ether, polydimethylsiloxane.

The composition is applicable to a surface that is or can be applied to the RFID tag or to which the RFID tag is applied. The application can be done with conventional methods known per se chosen from among: brush, dip coating, spin coating, drop casting, doctor blading, knife coating, bar coating, spray coating, Langmuir-Blodgett deposition and slot die coating, methods all in themselves known.

Another object of the invention is to develop an electronic device or a component thereof which comprises or is a radio frequency identification tag (RFID), preferably a passive RFID.

Another object of the invention is the use of one or more compounds selected from those described in the general formula of U.S. Pat. No. 10,144,744 or of a composition comprising said one or more compounds for application on an RFID tag for measuring the temperature integrity of a product.

Another object of the invention is the use as mentioned above in a method for identifying and managing the perishability of products which, due to their perishability, must be stored at temperatures lower than their degradation temperature (for instance room temperature) and whose perishability is connected to exposure to temperatures higher than said degradation temperature for periods of time connected to their perishability at such an higher temperature.

Still another object of the invention is a product or a product package on which a radio frequency identification tag is applied which comprises, as active material to be interfaced to said RFID, one or more compounds selected from those claimed in claim 1.

Another object of the invention is to develop a method implemented by a computer or by a mobile device (scanner, mobile phone, personal digital assistant (PDA), smartphone, tablet, laptop) wherein the method is implemented by a processor and comprises the stages of:
i) Acquisition of sensor operational values
ii) Acquisition of control and threshold values of the product
iii) Acquisition of the sensor signal
iv) Evaluation of the progress level of thermal relaxation
v) Counting of measurements and history
vi) Assignment of the healthiness level of the product

Other objects of the invention are to develop a computer program comprising a code able to execute the steps from i) to vi) as defined in the present description and in the claims when executed on a computer, and to develop a computer support comprising such a plan.

Further objects, features and advantages of the present invention will become apparent from the following detailed description, together with the accompanying drawings. It is understood that the detailed description and specific examples, while indicating preferred embodiments of the invention, are provided for illustrative purposes only, as changes and modifications in the spirit and scope of the invention will become apparent to those skilled in the art from the detailed description below.

### BRIEF DESCRIPTION OF THE DRAWINGS

For a better understanding of the various embodiments described herein, and to show more clearly how these various embodiments can be implemented, reference will be made, by way of example, to the accompanying drawings which show at least one exemplary embodiment. The accompanying drawings are illustrative of embodiments as exemplified herein and are not intended to limit the scope of the invention as identified by the claims.

FIGS. 1A-1B. Schematically illustrate a first and a second embodiment of an RFID tag (FIG. 1A), with the corresponding possible RFID readers, a smartphone and an RFID reader (FIG. 1B), which can be used to implement the present invention.

FIG. 2. Schematically illustrates the molecular structure of the ‘Photochromic Torsional Switch (PTS)’ architecture.

FIG. 3. Schematically illustrates the synthesis of a PTS unit not described in Chem. Sci. 2017, 8, 361-365 and in patent U.S. Pat. No. 10,144,744.

FIG. 4. Schematically illustrates synthetic methods for functionalization of PTS units.

FIG. 5. Schematically illustrates the general synthetic methods for the synthesis of the preferred compounds of the invention, detailed synthesis in the EXAMPLES section.

FIGS. 6A-6B. Show an assay of planar/cis to non-planar/trans (or conductive-to-non-conductive) conversion times (thermal relaxation) via UV-Vis absorption spectra. By way of example, in (FIG. 6A) the spectra of PTS tetrathiophene (compound 6) and in (FIG. 6B) those of PTS hexathiophene (compound 7) are reported.

FIGS. 7A-7C. Schematically illustrate a further exemplifying and non-limiting embodiment of the RFID tag according to the invention. (FIG. 7A) deposition of a coating containing PTS on the whole antenna (front view and top view); (FIG. 7B) deposition of a PTS containing coating on a portion of the antenna (front view and top view); (FIG. 7C) deposition of a PTS containing coating which joins the microchip to the antenna (front view and top view).

FIG. 8. Shows a block diagram of the steps of the operational method of a software according to the present invention.

FIGS. 9A-9B. Show the monitoring of the variations of the radio signals of a temperature integrity sensor according to the invention. Hexathiophene PTS (compound 7) is shown representatively. (FIG. 9A) Signal at 0° C.: 1) Unprocessed RFID (25° C.); 2) RFID fully coated with non-activated PTS materials (25° C.); 3) RFID with activated PTS materials (25° C., time 0); 4-7) RFID with activated PTS materials (0° C., time 24 h, 48 h, 72 h, and 96 h respectively). (FIG. 9B) Signal at room temperature (25° C.): 1) untreated RFID; 2) RFID fully coated with PTS materials; 3) RFID with activated PTS materials (time 0); 4) RFID with PTS materials in conductive-to-non-conductive conversion (time 12 h); 5) RFID with PTS materials in conductive-to-non-conductive conversion (time 24 h).

FIG. 10. Shows a table with preferred PTS compounds.

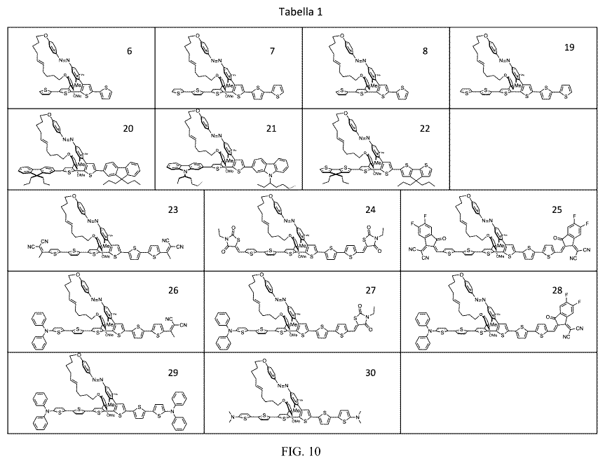

### DETAILED DESCRIPTION OF THE EMBODIMENTS

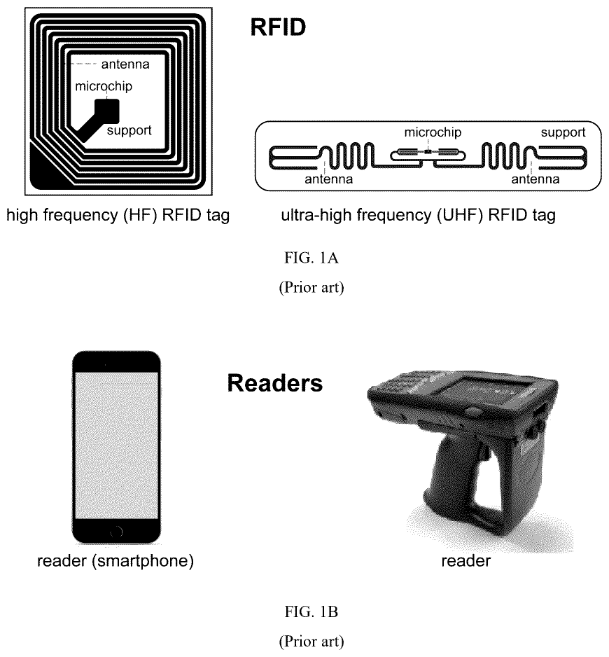

In the context of the present invention the following definitions are given:
the term “thermoresponsive” as used herein indicates a product or compound that responds to a change in temperature by modulating its chemical-physical properties, without undergoing deterioration.
the term “temperature integrity” means the continuity and conformity of the temperature in a given time interval, capable of guaranteeing the integrity of a product transported through the cold chain.
the words “temperature integrity sensor” and “temperature integrity seal” mean a device that checks the continuity and conformity of the temperature in a given time interval, so as to guarantee the integrity of a product transported through the cold chain.
the term “molecular actuator” means a “molecule” or a “part of a molecule” that transforms an external stimulus, e.g. light, in a mechanical motion at a molecular level, e.g. torsion and/or rotation along a molecular bond, which in turn induces a change to a more complex molecular system.
the term “molecular switch” means a “molecule” or a “part of a molecule” that can alter its isomeric state in a reversible way as a response to an external stimulus, e.g. light.
the term “π-conjugated system” means a system of p orbitals arranged in the same plane with delocalized electrons. Conjugated systems consist of an alternation of single bonds and multiple bonds.
the term “monomer” means a “molecule” having functional groups which allow it to combine several times with others to form a macromolecule, an oligomer or a polymer.
the term “oligomer” means a polymer formed by a limited number of monomers which can vary from 2 to 10 units.
the term “polymer” means a macromolecule of high molecular weight formed by a large number of monomers which can vary from 11 to 1000 units.
the term “sub-unit” or “aromatic sub-unit” means the aromatic cycle that constitutes a monomer, e.g. bithiophene as monomer will contain two sub-units of thiophene, fluorene as monomer consists of two sub-units of phenyl.
the term “PTS unit” indicates a monomer consisting of a bithiophene unit and an azobenzene unit, wherein the two thiophene sub-units are both connected to the two ends of the azobenzene: one by direct bond, the other by means of a carbon chain.
the term “PTS compound/s” means a molecule, an oligomer or a polymer containing at least one PTS unit in its structure. PTS compounds are therefore primarily light-responsive semiconductors. Such PTS compounds may also be referred to as “π-conjugated compounds responsive to light and temperature” and will simply be referred to as “thermoresponsive compounds” in the text.
the terms “activated” and “non-activated” material mean a solid state material made up of PTS compounds, or a mixture thereof, with additives, which has been irradiated with wavelengths of about 360 nm (activated) or that has not been exposed to lighting (not activated).
the term “vehicle” means a solvent (a liquid) which dissolves a solid solute giving rise to a solution.
the term “additive” means a chemical substance (or molecule) which improves the physical properties (e.g. mechanical, optical, electronic, etc.) of materials.
the words “radio frequency identification tag” and “RFID” mean a remote recognition, validation and/or storage of information technology which typically uses 5 frequency bands: low frequency (LF) (125-134, 2 kHz), high frequency (HF) (13.56 MHz), ultra high frequency (UHF) (865-956 MHZ) and microwave frequency (2.4 GHz and 5.8 GHz) corresponding to a approximative reading range respectively less than 0.5 meters, 1.2 meters, 5-10 m and finally up to 10 meters. The “tags” are simple resonant electrical circuits comprising inductive (L), capacitive (C) and resistive (R) elements on a plastic substrate (FIGS. 1A-1B).
the words “Passive RFID” mean a device without power, e.g. batteries, which uses the energy coming from the RFID reader (reader) for its operation.
the term “RFID reader” or “reader” means a powered device, consisting of a transceiver which is controlled by a microprocessor which has the task of requesting and receiving information in response from a tag. The reader puts the data from the RFID antennas in communication with the managing system.
the term “heterojunction” indicates the interface between semiconductor materials having different energy gaps and potential barriers for electrons and for holes. Specifically, this invention refers to the interface between organic semiconductors and inorganic semiconductors.
the term “about” indicates a variation of +/−10% for the values indicated.

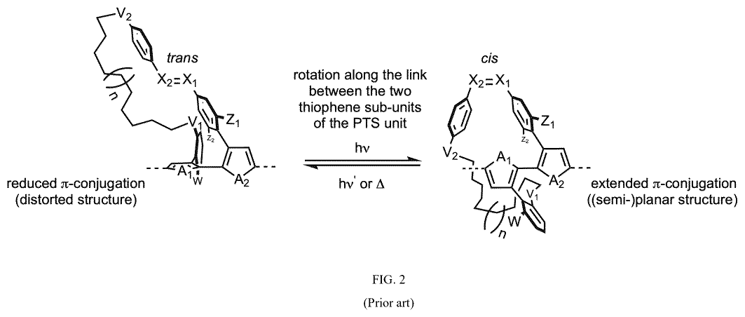

The semiconductors used as temperature integrity sensors coupled to a radio frequency identification tag (RFID), preferably a passive RFID, are based on the “Photochromic Torsional Switch” (PTS) architecture, which allows π-conjugated organic compounds to regulate their optical and electronic properties by means of light stimuli, a non-limiting example of these compounds is illustrated in FIG. 2.

It is worthwhile to initially point out that:
photochromic compounds do not work as organic semiconductors
organic semiconductors are not photochromic
PTS derivatives are both semiconductors and photochromic compounds.

The compounds used in the present invention are known and described in U.S. Pat. No. 10,144,744 and can be represented by the following general structural formula (I) whose substituents are also described in U.S. Pat. No. 10,144,744:

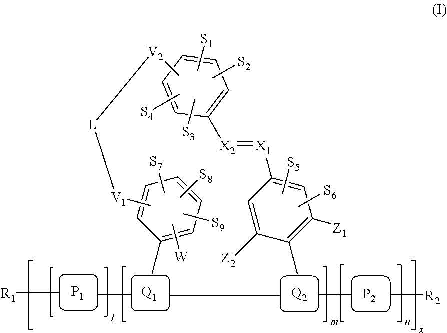

Said compounds are known as components of electronic instrumentation, in particular optical, electronic or electro-optical devices, such as for instance photochromatic photovoltaic devices, organic multicolor light emitting diode devices or field effect photo-tunable organic transistors. But they have never been used as temperature detectors, in particular they have never been used or described as temperature integrity (continuity) sensors in RFID radio frequency technology, especially in the field of passive RFID.

These compounds are designed to undergo a conformation change of their π-conjugated system via photoisomerization of a “molecular switch” orthogonally attached to the conjugation extension. The conformational variation of the π system thus makes it possible to vary the conductivity of the materials. These are therefore more conductive (conjugated π planarized/conductive system) (cis conformation) as a response to the absorption of light having about 360 nm wavelength. Conversely, they become less conductive (distorted/poorly conductive conjugation) (trans conformation) when they absorb light having a lower wavelength (e.g. about 240-260 nm, preferably 254 nm). Furthermore, the compounds spontaneously revert over time from the more conductive cis conformation to the less conductive trans conformation, by means of their thermal relaxation. This phenomenon is irreversible, unless the trans conformation is irradiated again and it is precisely this natural phenomenon that is exploited to produce the temperature continuity sensors according to the invention. Thermal relaxation can be blocked or slowed down at low temperatures, therefore the temperature continuity sensors according to the invention are suitable for detecting whether a pre-set temperature has remained constant or has varied until it reaches undesirable levels. The times required for thermal relaxation can vary from seconds(s) to hours (h) on the basis of the functional groups inserted in the azobenzene and the expert in the art, on the basis of the teachings of the following invention and his knowledge, will be able to identify the most suitable substituents and/or functional groups for the specific use for which the RFID is intended.

PTS semiconductors are particularly advantageous when used in passive RFID devices as an alternative to complex circuitry for low temperature (−5÷0° C.) integrity (continuity) control in the cold chain. The conformation changes of the PTS semiconductors, between their conductive and non-conductive form, are facilitated at room temperature (20÷25° C.) and blocked at low temperatures (−5÷0° C.). Thus, the changes in conductivity as the temperatures of the PTS semiconductors variation will be read as changes in the radio frequencies of the RFID devices. The RFID devices thus obtained will therefore be useful for measuring whether or not there has been continuity of temperature over time with respect to a predetermined value, or whether an undesired increase of it has occurred. Therefore, the invention relates to a device or temperature sensor which allows to detect whether the object to which said sensor is applied has been kept at the desired temperature or if there has been a rise in it, causing an undesired variation of the temperature itself.

The temperature continuity sensor or device comprises an RFID which comprises a support on which a thermoresponsive compound characterized by a π-conjugated system is applied, whose conformational variation varies the conductivity of the compound which is more conductive in response to an absorption of light wavelength of about 360 nm to a less conductive state thereof in response to absorption of shorter wavelength light. Said heat-responsive compound can be applied to the support in its least conductive conformation (typically at room temperature) and subsequently irradiated to make it more conductive or, alternatively, it can be applied directly to the support in its most conductive conformation at a temperature lower than 0° C. In both embodiments it will spontaneously and irreversibly pass from the more conductive conformation to the less conductive conformation as the temperature rises from 0° C. to room temperature.

The preferred compounds of the invention, also referred to simply as PTS, have the following general formula (II):

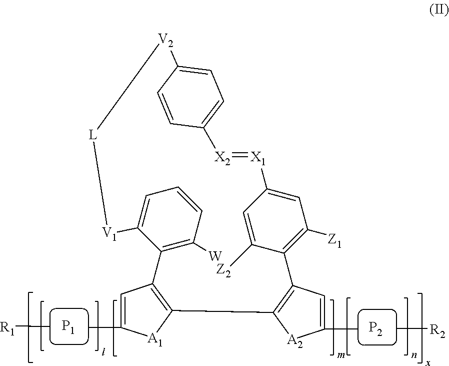

Wherein:
L=is a hydrocarbon chain with 5-15 carbon atoms wherein up to 3 hydrocarbon moieties (—CH₂—) can be replaced by one of the following moieties: O, NR_{L,1}, S, and/or wherein there can be up to 3 double or triple bonds, wherein the pairs of the type —CH₂—CH₂— are replaced by —R_{L,2}C=CR_{L,3}—, or —N═CR_{L,4}—, with the residues R_{L,1}—R_{L,4} independently selected from the group consisting of H, methyl, ethyl, propyl, isopropyl or benzyl;
W=is selected from the group consisting of: H, halogen, SR_{W,1}, methyl, ethyl, OR_{W,2}, COOR_{W,3}, with the residues R_{W,1}—R_{W,3} selected independently of each other, from group consisting of H, methyl, ethyl, propyl, isopropyl, butyl, pentanyl, hexanyl, heptanyl, octanyl, phenyl or benzyl;
V₁ and V₂=are selected independently of each other from the following groups: CH₂, S, O, NH, COO, CO, CONR_{V,1}, with the residues R_{V,1} selected from the group consisting of H, methyl, ethyl, propyl, isopropyl, phenyl or benzyl;
A₁ and A₂=are selected, independently of each other, from the following groups: S, O, NH, NR_{A,1}, BH, BR_{A,1}, PR_{A,1}, PR_{A,1}R_{A,2}, Se, CH═CH, CH═N, CH=PR_{A,1}R_{A,2}, CH₂, C—O, C═CH₂, C—CR_{A,1}R_{A,2}, with residues R_{A,1} and R_{A,2} selected, independently of each other, from the group consisting of H, methyl, ethyl, propyl or benzyl;
X₁ and X₂=are selected, independently of each other, from the following groups: N, CH;
Z₁ and Z₂=are selected independently of each other from the following group: halogen, methyl, ethyl, propyl, isopropyl, phenyl and benzyl, SR_{Z,1}, OR_{Z,2}, COOR_{Z,3}, NR_{Z,4}R_{Z,5}, NO₂, —CN, —SO₃H, with the radicals R_{Z,1}—R_{Z,5} independently selected from the group consisting of H, methyl, ethyl, propyl, isopropyl, benzyl, and primary, secondary and tertiary amines, both alkyl and phenyl;
P₁ and P₂=are one or a mixture of π-conjugated aromatic units which can independently of each other be selected from thiophene and its derivatives, ethylenedioxythiophene, indacenodithiophene, cyclopentadithiophene, phenyl and its derivatives, phenylvinylene, fluorene, indenofluorene, pyrene, pyrrole and its derivatives, pyrrol-2-one, maleimide, 2,1,3-benzothiadiazole, dithienylpyrrole, azole, diazole, triazole, tetrazole, indole, carbazole, or other groups selected from those indicated in scheme (I) below, which in turn can be substituted or unsubstituted, in the case of substitution R₃ can preferably be a hydrogen, halogen, methyl, ethyl, propyl, isopropyl, benzyl, phenyl, methoxy, ethoxy, propoxyl, butoxyl, pentoxyl, hexaneoxy, heptanoxy, octanoxyl, 3-methylheptane, 3-methoxyheptane, 7-methylpentadecane, 7-methoxypentadecane;

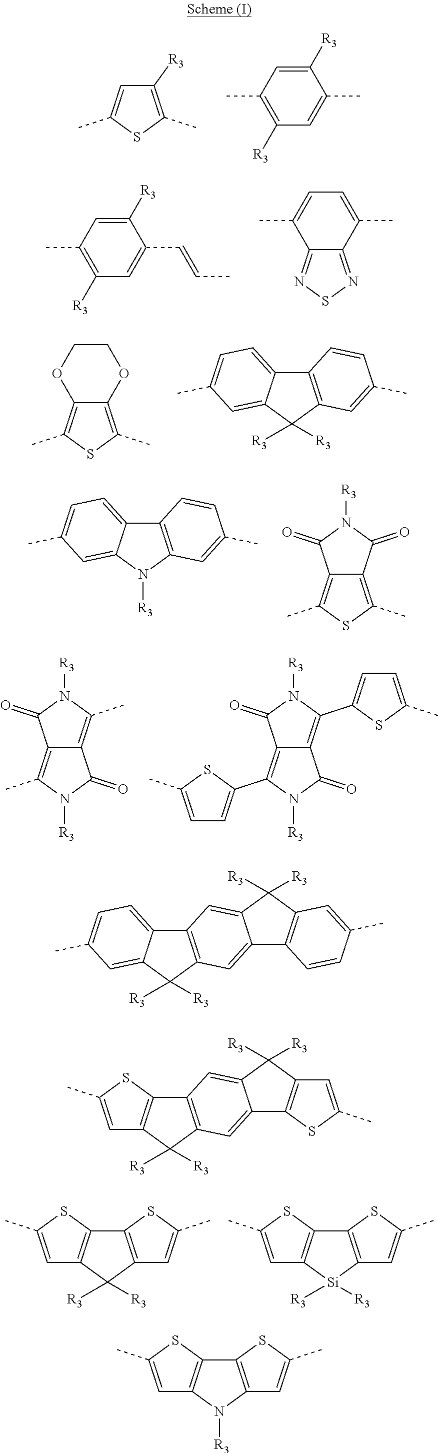

with
R₁ and R₂=are selected, independently of each other, from the following group: H, halogen, methyl, Sn(R_{R,1})₃, B(OH)₂, OR_{R,2}, NR_{R,3}R_{R,4}, NO₂, COOR_{R,5}, COR_{R,6}, S_{R,7}, —CN, —CCR_{R,8}, —SO₃H, —CR_{R,9}=C(CN)₂, —CR_{R,10}=C(CN)(COOR_{R,11}), —C(CN)—C(CN)₂, methyldicyanovinyl, substituted N-ethylrhodanine and its derivatives, substituted and unsubstituted 3-(dicyanomethylidene) indan-1-one and its derivatives, substituted and unsubstituted ferrocene and its derivatives, substituted and unsubstituted pyridine and its derivatives, comprising pyridine groups, pentafluorophenyl, substituted and unsubstituted fullerene and its derivatives, both alkyl and phenyl primary, secondary and tertiary amines; the residues R_{R,1}—R_{R,11} are independently selected from the group consisting of: methyl, ethyl, propyl, isopropyl, phenyl, benzyl and both alkyl and phenyl primary, secondary and tertiary amines;
and wherein:
l=is selected to be an integer between 0 and 10, preferably 1, or 2, or 3, or 4, or 5, or 6, or 7, or 8, or 9;
m=is selected to be an integer between 1 and 10, preferably 2, or 3, or 4, or 5, or 6, or 7, or 8, or 9;
n=is selected to be an integer between 0 and 10, preferably 1, or 2, or 3, or 4, or 5, or 6, or 7, or 8, or 9;
x=is selected as an integer between 1 and 10,000, preferably between 1 and 10, more preferably between 2 and 10
with the proviso that 1+n is at least 1.

Particularly preferred compounds of the invention are compounds which have in the molecule a tetra-thiophene group (formula (III)), or hexa-thiophene (formula (IV)), or fluorene-thiophene (formula (V)), or carbazole-thiophene (formula (VI)), or cyclopentadithiophene-thiophene (formula (VII)) combined with an azobenzene which acts as a molecular switch through light stimuli.

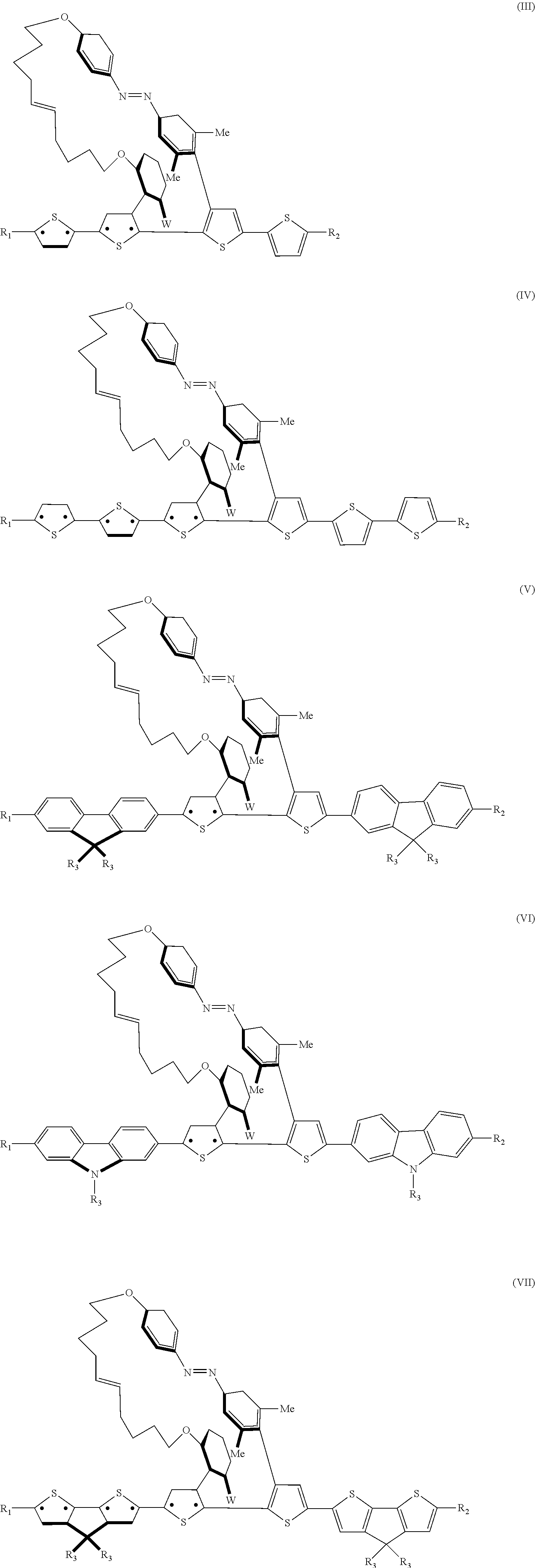

Wherein:
R₁, R₂=are selected as described above;
R₃=is selected as described above;
W=is selected as described above;

The range of action of compounds (III), (IV), (V), (VI) and (VII) is typically in the order of hours (h), with a maximum time of about 36 h for the complete transition from cis to trans. Among the compounds (III), (IV), (V), (VI) and (VII), the following compounds are particularly preferred, having relaxation times between 20 and 40 hours:

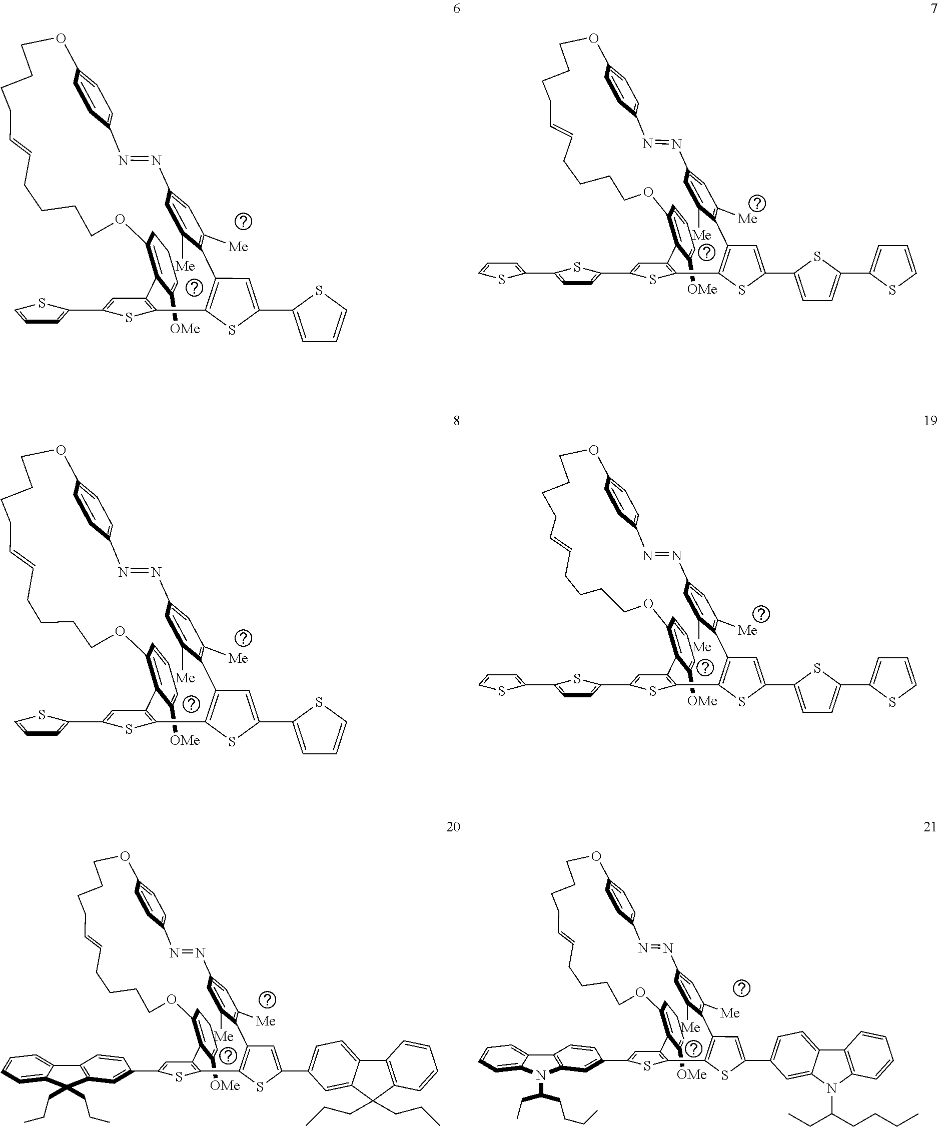

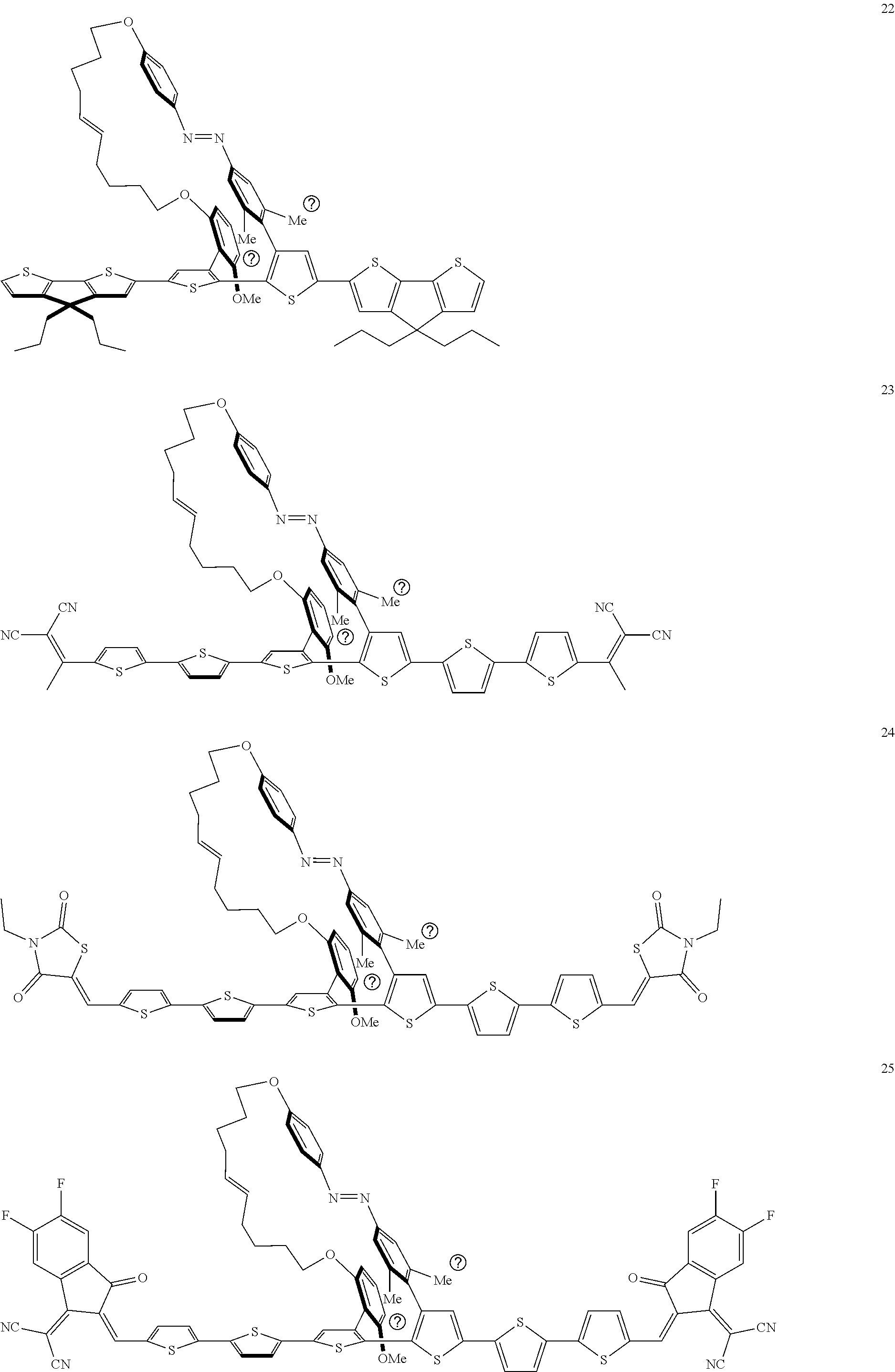

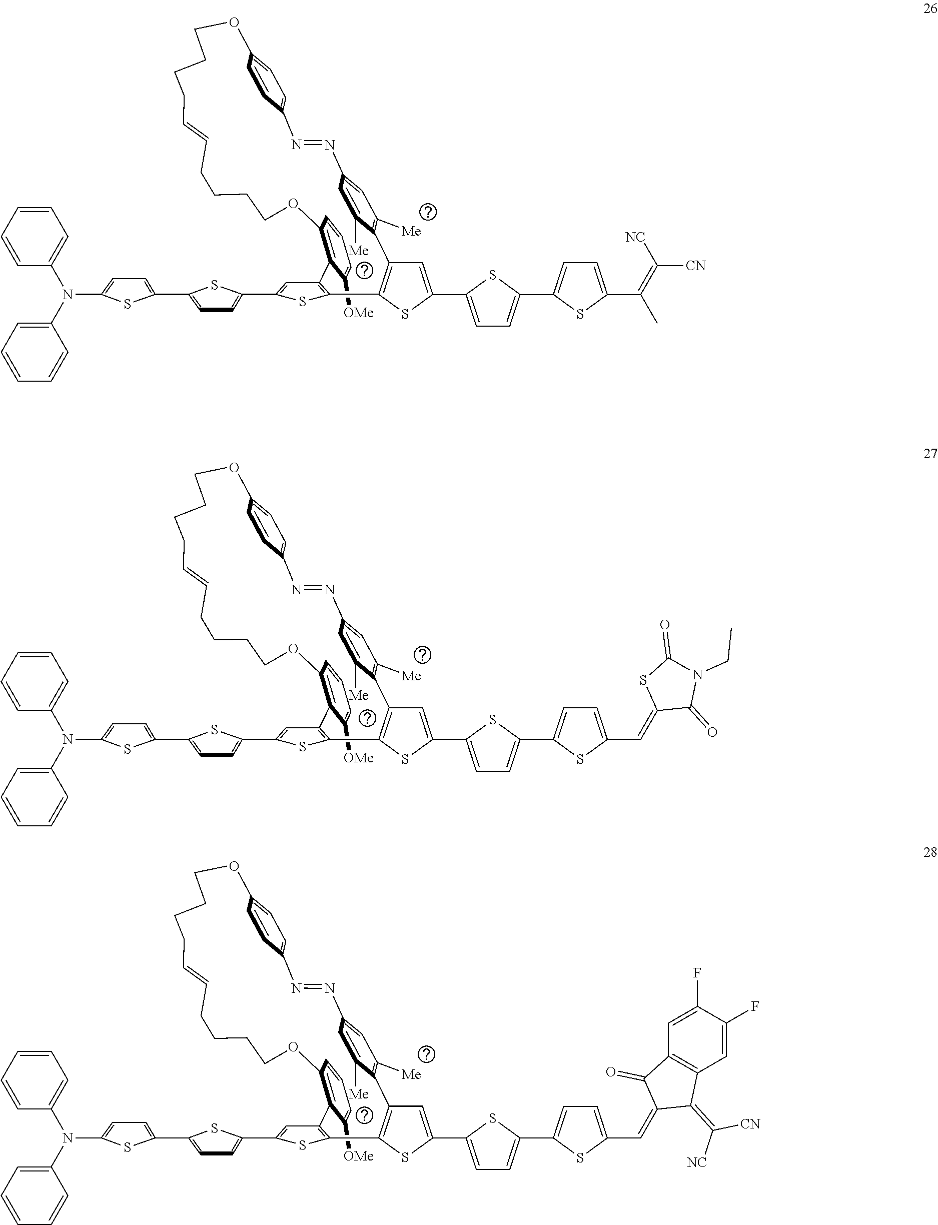

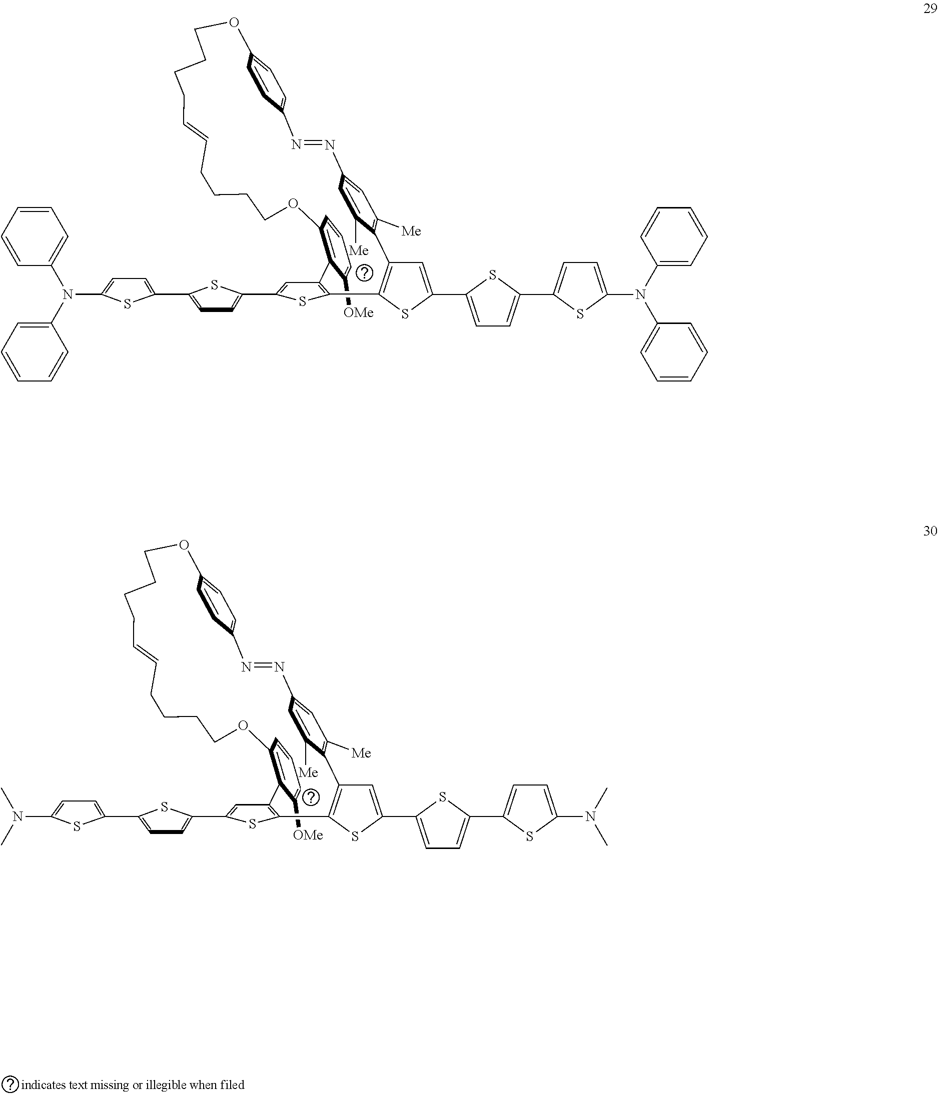

The maximum transition time indicates the length of time a product can be monitored by the temperature integrity sensor. More specifically, with the above molecules, the Temperature Integrity Sensor can monitor any product that, if exposed to incorrect storage temperatures, could deteriorate within minutes, hours and days (no longer than transition maximum, e.g. 1.5 days). The molecules based on the PTS architecture to be integrated with the RFID antenna will then be chosen on the basis of their cis-to-trans transition time in order to ensure that the product deterioration time is not longer than that of the cis-to-trans transition.

The PTS compounds can be applied on the tag as a diluted solution with per se known conventional methods chosen among: brush, dip coating, spin coating, drop casting, doctor blading, knife coating, bar coating, spray coating, Langmuir-Blodgett deposition and slot die coating. The particularly preferred method of the invention is dropcasting. The solvent will then be removed by evaporation, with or without reduced pressure, possibly assisted by heating by means of a heating plate at temperatures ranging from 30° C. to 100° C.

The usable solvents can be aprotic non-polar solvents such as toluene, tetrahydrofuran, 2-methyl-tetrafuran, 1,2,4-trimethylbenzene, o-xylene, anisole; or halogenated solvents such as chlorobenzene, chloroform, dichloromethane, 1,2,4-trichlorobenzene; 1,2-dichlorobenzene, mixtures thereof and solvents with similar characteristics.

Diluted solutions are prepared, for instance, but not limited to, by weighing 1 to 10 portions (mg) of the PTS compounds per 1 volume portion (mL) of solvent and/or carrier.

The compounds can be colored and have a different coloration between the planarized (cis switch) and distorted (trans switch) forms and this color variation can be used, for instance, in such a way as to thus make the temperature transition evident. Alternatively, any color variation can be concealed by covering the RFID antenna to prevent tampering aimed at returning the product to its initial temperature conditions.

The RFID can be associated with a sound and/or light signal to indicate that the product on which the antenna is applied has had a discontinuity (increase) in temperature which causes its deterioration.

According to a further embodiment, the invention relates to an RFID tag device which can be shaped as follows.

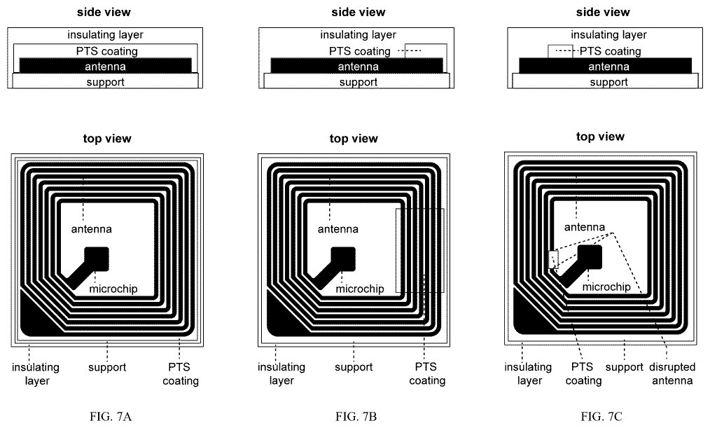

A non-limiting schematization is represented in FIGS. 7A-7C wherein are represented:
A support, usually plastic or paper, which bears
the metal antenna generally made of aluminum or copper or conductive ink (e.g. based on silver or carbonaceous particles).
A PTS-based composition is deposited on the antenna or on portions of it.
Everything is preserved by a light insulating layer (typically plastic or paper), specifically, insulating at wavelengths ranging from about 250 nm to about 400 nm.

PTS semiconductor compounds can be deposited in thin film form on the RFID tag antenna. The film is then activated by irradiating for a few seconds or minutes (based on the molecules used) with an appropriate wavelength, for instance from about 350 nm to about 360 nm, so as to convert the PTS semiconductors from the initial non-conductive (or less conductive) trans form to the conductive cis-one (tag activation).

Once the film of PTS materials is activated, the insulating layer is applied, and the label can be stored in a refrigerator or freezer, or applied directly to the product to be monitored.

The organic layer containing the PTS architecture, deposited and activated on the antenna, will result in providing a distinct and different radio signal from that of the non-functionalized antenna (without PTS materials), as some of the intrinsic physical properties of the RFID circuit are changed, e.g. the frequency, impedance (Z) and reflection coefficient (S11).

For instance, if the device is stored at low temperatures (−5÷0° C.), the radio signal obtained after the activation of the PTS compounds will remain unchanged. On the contrary, with the gradual increase in temperature the PTS molecules in their conductive cis form will gradually convert into their non-conductive (or less conductive) trans form, thus varying in a distinct and recordable by a reader way the frequency and/or the impedance (Z) and/or reflection coefficient (S11) of the device during the process.

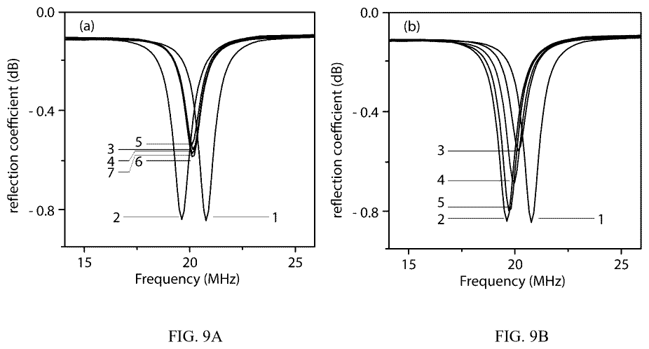

When the reader interrogates the device, the response radio signal will be characteristic depending on the state of progression of the conformation assumed by the material (cis conductive and trans non-conductive) (see FIGS. 9A-9B).

Thus, the device according to the invention provides an irreversible record of specific temperature events to which the indicator (i.e. the PTS compound) applied on the device has been exposed. Then the PTS temperature indicator will be associated with the perishable host product to monitor the host product's exposure to temperatures above the safe temperature to maintain it intact. In that the activated PTS compound is configured to undergo a conformational change, and consequently also a conductivity change, in response to exposure to a temperature above a storage temperature, which is typically below 0° C. The variation in conductivity, and therefore the response radio signal of the device, may occur over a period of time ranging from minutes to days, however preset for the specific product to be monitored.

The advantage of the device of the invention is that of using PTS molecules which are characterized by a spontaneous, unidirectional and irreversible variation of the molecular conformation, which in turn results in a change in conductivity, since the modified conductivity will persist after the PTS indicator material is no longer exposed to temperatures above the storage temperature. Therefore, after a variation in conductivity and after a subsequent exposure to a temperature lower than the storage temperature, the PTS indicator material will maintain the modified conductivity allowing to record the temperature alteration to which the product to be monitored has been subjected.

According to a further embodiment, the invention relates to an electronic system such as a computer and implemented by software for managing the control and detection of the temperature continuity of products subject to deterioration if subjected for a certain period of time at temperatures higher than those indicated as optimal for their preservation. Optimum temperatures, temperatures above optimum temperatures, and times to exceed optimum temperatures vary from product to product and are determined by the manufacturer of the perishable product.

Perishable products are all edible products sold in the food distribution chains of refrigerated and frozen products, pharmaceutical products to be kept at temperatures below −5° C.-+5° C. such as a vaccine, or an antibiotic; or a biological-medical product such as a sample of a body fluid or tissue, or an organ.

The method is meant to manage the expiration of products, for instance a method to be used by operators in the points of sale of refrigerated/frozen products and by all operators (such as, for instance, operators in the health sector) who need to acquire data on the storage of a product at a temperature constantly below the degradation temperature.

The method, which can be implemented through an electronic system such as a computer, combined with the RFID device of the invention allows to guarantee the maintenance of the optimal preservation temperature beyond the indication of the expiry date, as long as the RFID device is alone, is combined with the detection method, it allows to detect if the product has been properly stored and the cold chain has been maintained without alterations or tampering.

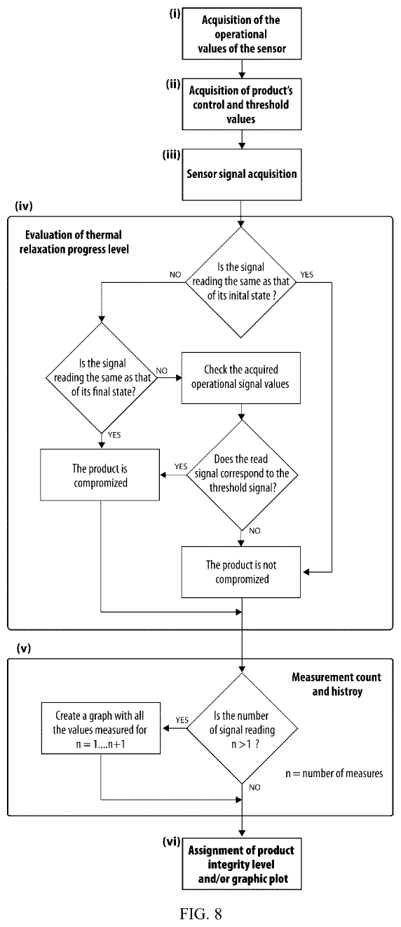

The method involves the following process stages, rationalized in the flow chart illustrated in the attached FIG. 8.

The method for measuring the temperature integrity of a product comprises the following steps:
i) Acquisition of sensor operational values comprising:
   The PTS coated RFID compounds radio signal before and after activation with light,
   the transition times (thermal relaxation) from the “cis conductive” to the “trans non-conductive” conformation at different temperatures between −5÷0° C. to 50° C. with intervals of 1÷5° C.
These values will be measured and entered into the method to manage the expiry of the products;
ii) Acquisition of control and threshold values of the product comprising:
   the optimal storage temperature values,
   the critical temperature threshold at which the product starts to deteriorate or enters the “danger zone”,
   the maximum critical temperature time to which the product can be exposed before it starts to deteriorate or enters the “danger zone”,
These values will be acquired by the supplier of the product subject to monitoring and inserted in the method for managing the expiry of the products;
iii) Acquisition of sensor signal:
   The preservation status of the “cis-conductive” conformation of the PTS compounds will be checked by means of an RFID reader, whose radio signal will be acquired and inserted into the method for managing the expiry of the products;
iv) Evaluation of the level of progress of the thermal relaxation that will be performed by means of a comparison between:
   the radio signal read by the reader (iii),
   the control and threshold values of the product (ii), e
   the operational values of the sensor (i);
v) Counting of measurements and history in order to generate a history of the number of measurements made and the relative acquisition times;
vi) Assignment of the healthiness level of the product.
The preservation status of the product will be shown with three alert levels:
   “intact product”, preservation at low temperatures was respected throughout the time (final signal from the antenna equal to the initial signal),
   “product in good condition”, storage at low temperatures has been respected for most of the time (the final signal from the antenna is different from the initial signal but also different from the signal corresponding to reaching the threshold temperature);
   “compromised product”, storage at low temperatures has not been respected (the final signal from the antenna is different from the initial signal but equal or similar to the signal corresponding to reaching or exceeding the threshold temperature).

The method can be implemented through a computer program comprising a code suitable for performing steps i) to vi) as defined above when executed on a system comprising an RFID, such as for instance a computer, preferably a computer, a tablet, a smartphone or equivalent system.

Another object of the invention is a computer support comprising the computer program as defined above.

The conductive-to-non-conductive conversion process by means of thermal relaxation is unidirectional. Therefore the conductivity state can be maintained/stopped at low temperatures (T=−5° C.−0° C.) and resumes its course at any time from when the tag will be exposed to higher temperatures (T>5° C.) of the cooling ones (stop the process). The integrity (continuity) of low temperatures in the cold chain will thus be recorded as a radio frequency variation, induced by the progress of the conductive-to-non-conductive conversion of PTS semiconductors. In this way, if a product is stored even temporarily outside the storage temperatures, the history of the freezing-thawing-refreezing process will be recorded as a variation of the radio signal.

The unidirectional aspect is advantageous for verifying that the cold chain is maintained, precisely because the process is thermodynamically irreversible, i.e. the PTS unit does not return to its original state with the aid of low temperature alone. This makes it possible to extend the scope of the proposed technology to other fields such as anti-counterfeiting. At the same time, the conversion from conductive-to-non-conductive of PTS semiconductors is spontaneous, thus allowing to perform a working activity on an electronic circuit without the aid of a power supply (self-powered). Furthermore, the preparation process of the temperature integrity seal is compatible or easily integrated with the current production tagging processes, allowing to limit both the production costs of the device and to extend the field of use of the technology proposed as a possible component of the ‘“internet of things” (IoT).

The following examples are illustrative of the invention and are not to be considered as limiting its scope.

### Methods

### Synthesis of PTS-Based Compounds.

The synthesis of the PTS 1 unit is already known and described both in U.S. Pat. No. 10,144,744 and in Chem. Sci., 2017, 8, 361. We therefore report the procedures for the synthesis of the PTS 2 unit and those for the functionalization of the PTS 1 and 2 units. The functionalized versions of the PTS units are the basis for the preparation of oligomers and polymers, therefore we also report here the general procedures for the synthesis of compounds containing the PTS unit as a monomer.

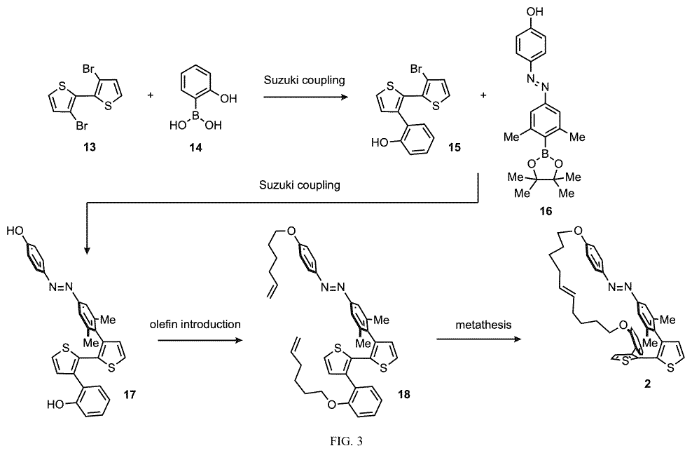

### General Procedure for PTS 2 Unit Synthesis, FIG. 3.

Compound 15: A mixture containing 3,3′-dibromo-2,2′-bithiophene (compound 13) (1.0 g, 3.09 mmol), hydroxyphenyl acid 14 (0.51 g, 3.70 mmol), Pd(PPh₃)₄ (0.71 g, 0.62 mmol), and K₂CO₃ (1.71 g, 12.34 mmol) is dissolved in a dioxane/water mixture (19 mL/1 mL) under an argon atmosphere. The mixture is stirred and heated to 80° C. for 48 hours, then allowed to reach room temperature. The reaction is then diluted by the addition of ethyl acetate, and the mixture transferred to a separating funnel to be washed with brine. The organic phase is then dried using MgSO₄ and the organic solvent evaporated under reduced pressure. The final product is finally purified by silica chromatography (eluent: petroleum ether:dichloromethane 1:1) to give 0.53 g (51%) of compound 15 as a white solid. ¹H NMR (400 MHZ, CDCl₃): δ=7.59 (d, J=5.2 Hz, 1H), 7.26 (t, J=8.4 Hz, 1H), 7.24 (d, J=5.3 Hz, 1H), 7.18 (d, J=5.2 Hz, 1H), 7.14 (dd, J=7.5, 1.2 Hz, 1H), 6.98 (d, J=5.4 Hz, 1H), 6.95-6.89 (m, 2H), 5.03 (s, 1H).

Compound 17: A mixture containing compound 15 (0.53 g, 1.56 mmol), compound 16 (1.09 g, 3.11 mmol), Pd(PPh₃)₄ (0.36 g, 0.31 mmol), and K₂CO₃ (1.08 g, 7.79 mmol) is dissolved in DMF (5 mL) under an argon atmosphere. The mixture is stirred and heated to 95° C. for 48 hours, then allowed to reach room temperature. The reaction is then diluted by the addition of ethyl acetate, and the mixture transferred to a separating funnel to be washed with brine. The organic phase is then dried using MgSO₄ and the organic solvent evaporated under reduced pressure. The final product is finally purified by silica chromatography (eluent: dichloromethane:ethyl acetate 9.5:0.5) followed by a second column chromatography (eluent: petroleum ether:acetone 7:3) to give 0.31 g (41%) of compound 17 in the form of a yellow solid. ¹H NMR (400 MHZ, CDCl₃): δ=7.97 (d, J=8.1 Hz, 2H), 7.64 (s, 2H), 7.33 (t, J=7.2 Hz, 1H), 7.23 (d, J=5.2 Hz, 1H), 7.10 (dd, J=7.5, 1.4 Hz, 1H), 7.02 (d, J=8.3 Hz, 2H), 6.99-6.94 (m, 3H), 6.92 (d, J=5.2 Hz, 1H), 6.81 (d, J=5.1 Hz, 1H), 4.91 (s, 2H), 2.08 (s, 6H).

Compound 18: A mixture containing 6-bromo-1-hexene (0.42 mL, 3.15 mmol), compound 17 (0.25 g, 0.52 mmol) and K₂CO3 (0.22 g, 1.57 mmol) is dissolved in DMF (5 mL). The mixture is stirred and heated to 95° C. for 48 hours, then allowed to reach room temperature. The reaction is then diluted by the addition of ethyl acetate, and the mixture transferred to a separating funnel to be washed with brine. The organic phase is then dried using MgSO₄ and the organic solvent evaporated under reduced pressure. The final product is finally purified by silica chromatography (eluent: petroleum ether:dichloromethane 1:1) to give 0.21 g (63%) of compound 18 as an orange solid. ¹H NMR (400 MHZ, CDCl₃): δ=7.95 (d, J=8.9 Hz, 2H), 7.56 (s, 2H), 7.31 (d, J=8.8 Hz, 1H), 7.24 (d, J=5.2 Hz, 1H), 7.11 (d, J=5.3 Hz, 2H), 7.04 (d, J=8.9 Hz, 2H), 6.96-6.84 (m, 3H), 6.78 (d, J=5.2 Hz, 1H), 5.95-5.81 (m, 1H), 5.82-5.69 (m, 1H), 5.15-4.82 (m, 4H), 4.10 (t, J=6.4 Hz, 2H), 3.80 (t, J=6.5 Hz, 2H), 2.24-2.07 (m, 2H), 2.04 (s, 6H), 2.02-1.95 (m, 2H), 1.92-1.82 (m, 2H), 1.74-1.46 (m, 4H), 1.41-1.27 (m, 2H)).

Compound 2 (PTS Unit 2): A mixture containing compound 18 (100 mg, 0.15 mmol) and 2nd gen. Grubbs' catalyst (25 mg, 0.03 mmol) is dissolved in anhydrous dichloromethane (800 mL) under an argon atmosphere. The mixture is stirred and heated at 40° C. for 6 hours, then allowed to reach room temperature. The mixture is then filtered through silica to remove the catalyst, and the organic solvent evaporated under reduced pressure. The final product is finally purified by silica chromatography (eluent: dichloromethane:petroleum ether 7:3) to give 50.9 mg (53%) of compound 2 as an orange solid. ¹H NMR (400 MHZ, THF-d8): δ=7.94 (d, J=8.8 Hz, 2H), 7.53 (d, J=5.2 Hz, 1H), 7.41 (s, 1H), 7.29 (d, J=5.2 Hz, 1H), 7.25 (s, 1H), 7.18-7.08 (m, 3H), 6.86 (d, J=5.2 Hz, 1H), 6.72 (d, J=5.2 Hz, 1H), 6.41 (d, J=8.3 Hz, 1H), 6.33 (d, J=8.3 Hz, 1H), 4.94 (t, J=3.2 Hz, 2H), 4.37 (t, J=5.0 Hz, 2H), 3.48 (s, 3H), 3.38-3.28 (m, 1H), 3.23-3.04 (m, 1H), 1.96 (s, 3H), 1.94-1.89 (m, 2H), 1.68-1.57 (m, 2H), 1.56-1.48 (m, 4H), 1.45 (s, 3H), 1.28-1.16 (m, 1H), 1.16-1.08 (m, 1H), 0.82-0.72 (m, 1H), 0.69-0.57 (m, 1H).

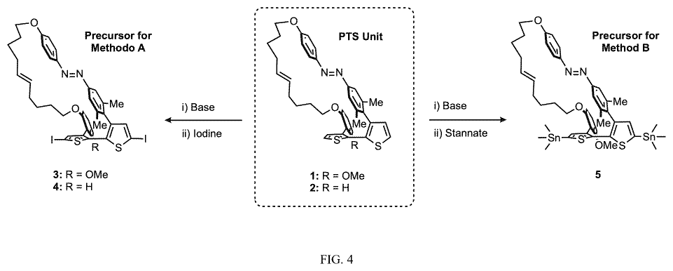

General procedure for the synthesis of the di-functionalized PTS unit Precursors for the Suzuki (Method A) and Stille (Method B) reactions, FIG. 4.

Compound 3: A solution of n-butyllithium in 1M hexane solution (0.41 mL, 0.641 mmol) is slowly added to a solution of diisopropylamine (0.099 mL, 0.705 mmol) in anhydrous THF (0.8 mL) at −78° C. under an argon atmosphere. The reaction mixture is left to reach the temperature of 0° C. and stirred for a further 10 minutes. The resulting lithium diisopropylamide (LDA) solution is slowly added to a solution of the PTS unit, compound 1, (104 mg, 0.160 mmol) in anhydrous THF (1.3 mL) at −78° C. The resulting mixture is stirred for about 3 hours at −78° C. Next, a solution of iodine in anhydrous THF (0.4 mL) is added to the reaction mixture at −78° C. The resulting reaction mixture is stirred for about 24 hours, and the temperature is allowed to slowly rise to room temperature. The reaction is quenched by the addition of a saturated NH₄Cl solution, the mixture transferred to a separating funnel to be washed with ethyl acetate and brine. The organic phase is then dried using MgSO₄ and the organic solvent evaporated under reduced pressure. The final product is finally purified by silica chromatography (eluent petroleum ether:dichloromethane 1:1) to give 87 mg (60%) of the desired product (compound 3) as a brown oil. ¹H NMR (400 MHZ, THF-d8): 8-7.90 (d, J=8.8 Hz, 2H), 7.40 (s, 1H), 7.20 (s, 1H), 7.13 (t, J=8.3 Hz, 1H), 7.07 (d, J=8.9 Hz, 2H), 7.03 (s, 1H), 6.83 (s, 1H), 6.39 (d, J=8.3 Hz, 1H), 6.29 (d, J=8.3 Hz, 1H), 4.93-4.88 (m, 2H), 4.36-4.30 (m, 2H), 3.50 (s, 3H), 3.33-3.26 (m, 1H), 3.15-3.07 (m, 1H), 1.97 (s, 3H), 1.93-1.88 (m, 2H), 1.63-1.55 (m, 2H), 1.54-1.46 (m, 4H), 1.38 (s, 3H), 1.24-1.18 (m, 1H), 1.12-1.04 (m, 1H), 0.76-0.70 (m, 1H), 0.65-0.58 (m, 1H).

Compound 4: A solution of n-butyllithium in 1M hexane solution (0.337 mL, 0.844 mmol) is slowly added to a solution of diisopropylamine (0.130 mL, 0.928 mmol) in anhydrous THF (1 mL) at −78° C. under an argon atmosphere. The reaction mixture is left to reach a temperature of 0° C. and stirred for a further 10 minutes. The resulting lithium diisopropylamide (LDA) solution is slowly added to a solution of the PTS unit, compound 2, (130 mg, 0.211 mmol) in anhydrous THF (1.8 mL) at −78° C. The resulting mixture is stirred for about 3 hours at −78° C. Next, an iodine solution (321 mg, 1.266 mmol) in anhydrous THF (0. mL) is added to the reaction mixture at −78° C. The resulting reaction mixture is stirred for about 24 hours, and the temperature is allowed to slowly rise to room temperature. The reaction is quenched by the addition of a saturated NH₄Cl solution, the mixture transferred to a separating funnel to be washed with ethyl acetate and brine. The organic phase is then dried using MgSO₄ and the organic solvent evaporated under reduced pressure. The final product is finally purified by silica chromatography (eluent petroleum ether:dichloromethane 7:3) to give 108 mg (59%) of the desired product (compound 4) as a brown oil. ¹H NMR (400 MHZ, THF-d8): 8-7.90 (d, J=8.8 Hz, 2H), 7.40 (s, 1H), 7.16 (s, 1H), 7.15 (d, J=10.4 Hz, 1H), 7.12 (s, 1H), 7.07 (d, J=8.8 Hz, 2H), 7.02 (s, 1H), 6.81 (d, J=7.3 Hz, 1H), 6.70 (t, J=7.4 Hz, 1H), 6.64 (d, J=8.3 Hz, 1H), 4.96-4.91 (m, 2H), 4.36-4.30 (m, 2H), 3.42-3.33 (m, 1H), 3.19-3.11 (m, 1H), 1.92 (s, 3H), 1.66-1.58 (m, 2H), 1.56-1.46 (m, 4H), 1.95-1.89 (m, 5H), 1.32-1.22 (m, 1H), 1.20-1.09 (m, 1H), 0.81-0.72 (m, 1H), 0.70-0.60 (m, 1H).

Compound 5: A solution of n-butyllithium in 1M hexane (0.17 mL, 0.421 mmol) is slowly added to a solution of diisopropylamine (0.065 mL, 0.463 mmol) in anhydrous THF (0.5 mL) at −78° C. under an argon atmosphere. The reaction mixture is left to reach the temperature of 0° C. and stirred for a further 10 minutes. The resulting lithium diisopropylamide (LDA) solution is slowly added to a solution of the PTS unit, compound 1, (68 mg, 0.105 mmol) in anhydrous THF (0.9 mL) at −78° C.). The resulting mixture is stirred for about 3 hours at −78° C. Next, a solution of trimethyl chloro stannane (126 mg, 0.631 mmol) in anhydrous THF (0.3 mL) is added to the reaction mixture at −78° C. The reaction mixture is stirred for about 24 hours and the temperature is allowed to slowly rise to room temperature. The reaction is then diluted by the addition of ethyl acetate, the mixture transferred to a separating funnel to be washed with brine. The organic phase is then dried using MgSO₄ and the organic solvent evaporated under reduced pressure. The desired product (compound 5) is obtained without further purification (93.8 mg, 98%), as a yellow solid. ¹H NMR (400 MHZ, THF-d8): 8-7.89 (d, J=8.8 Hz, 2H), 7.34 (s, 1H), 7.18 (s, 1H), 7.08 (t, J=8.3 Hz, 1H), 7.06 (d, J=8.9 Hz, 2H), 6.88 (s, 1H), 6.76 (s, 1H), 6.33 (d, J=8.3, 1H), 6.25 (d, J=8.3, 1H), 4.90 (t, J=3.1 Hz, 2H), 4.34-4.30 (m, 2H), 3.38 (s, 3H), 3.32-3.25 (m, 1H), 3.12-3.04 (m, 1H), 1.94-1.89 (m, 2H), 1.87 (s, 3H), 1.63-1.55 (m, 2H), 1.53-1.46 (m, 4H), 1.43 (s, 3H), 1.21-1.14 (m, 1H), 1.13-1.05 (m, 1H), 0.78-0.70 (m, 1H), 0.67-0.61 (m, 1H), 0.46 (s, 1H), 0.39 (s, 7H), 0.30 (s, 8H), 0.23 (t, J=5.0 Hz, 2H).

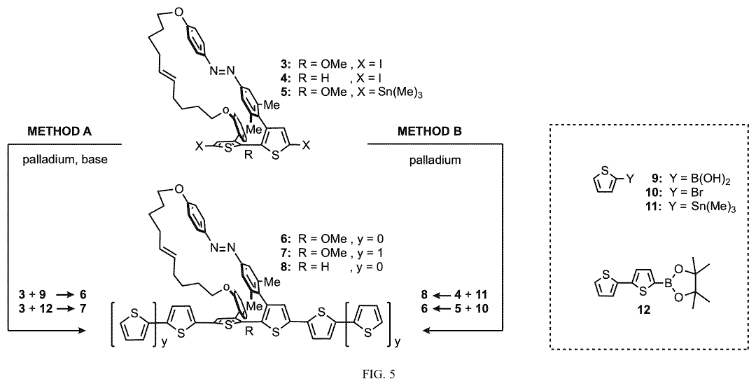

### General Procedures for the Synthesis of PTS Compounds by Means of the Suzuki Reaction (Method a) and the Stille Reaction (Method B), FIG. 5.

As a non-limiting example of Method A and Method B, the following procedures are contextualized to the syntheses of compounds 6, 7 and 8)

Method A, PTS quaterthiophene with OMe (compound 6): A mixture containing the PTS unit diiodate (compound 3) (30 mg, 0.033 mmol), thiophene-2-boronic acid 9 (21 mg, 0.0.167 mmol), Pd(PPh₃)₄ (7.6 mg, 0.007 mmol), and K₂CO₃ (23 mg, 0.166 mmol) are dissolved in DMF (1 mL) under an argon atmosphere. The mixture is stirred and heated to 85° C. for 18 hours, then allowed to reach room temperature. The reaction is then diluted by the addition of ethyl acetate, and the mixture transferred to a separating funnel to be washed with brine. The organic phase is then dried using MgSO₄ and the organic solvent evaporated under reduced pressure. The final product is finally purified by silica chromatography (eluent: petroleum ether:dichloromethane 1:1) to give 16.8 mg (62%) of compound 6 as an orange solid.

Method B, PTS quaterthiophene with OMe (compound 6): A mixture containing the unit PTS distannate (compound 5) (79 mg, 0.088 mmol), 2-bromothiophene 10 (42 μL, 0.437 mmol), Pd(PPh₃)₄ (20 mg, 0.018 mmol) is dissolved in DMF (2.6 mL) under an argon atmosphere. The mixture is stirred and heated to 85° C. for 15 hours, then allowed to reach room temperature. The reaction is then diluted by the addition of ethyl acetate, and the mixture transferred to a separating funnel to be washed with brine. The organic phase is then dried using MgSO₄ and the organic solvent evaporated under reduced pressure. The final product is finally purified by silica chromatography (eluent: petroleum ether:dichloromethane 1:1) to give 29 mg (41%) of compound 6 as an orange solid. ¹H NMR (400 MHZ, THF-d8): δ=7.91 (d, J=8.8 Hz, 2H), 7.42 (s, 1H), 7.34 (d, J=5.1 Hz, 1H), 7.30 (d, J=3.6 Hz, 1H), 7.26 (d, J=4.9 Hz, 1H), 7.23 (s, 1H), 7.16 (d, J=2.4 Hz, 1H), 7.14 (t, J=8.3 Hz, 1H), 7.07 (d, J=9.0 Hz, 2H), 7.04 (dd, J=5.5, 4.3 Hz, 1H), 7.02 (s, 1H), 6.98 (dd, J=5.0, 3.6 Hz, 1H), 6.85 (s, 1H), 6.41 (d, J=8.3 Hz, 1H), 6.31 (d, J=8.3 Hz, 1H), 4.93-4.88 (m, 2H), 4.38-4.29 (m, 2H), 3.53 (s, 3H), 3.38-3.29 (m, 1H), 3.19-3.09 (m, 1H), 2.05 (s, 3H), 1.94-1.87 (s, 2H), 1.63-1.55 (m, 2H), 1.52-1.44 (m, 7H), 1.30-1.20 (m, 1H), 1.17-1.08 (m, 1H), 0.80-0.70 (m, 1H), 0.69-0.57 (m, 1H).

Method A, PTS hexathiophene with OMe (Compound 7): A mixture containing the PTS unit diiodate (compound 3) (49.2 mg, 0.055 mmol), 2-([2,2′-bitiophen]-5-yl)-4,4,5,5-tetramethyl-1,3,2-dioxaborolane 12 (79.9 mg, 0.273 mmol), Pd(PPh₃)₄ (12.6 mg, 0.011 mmol), and K₂CO₃ (37.7 mg, 0.273 mmol) is dissolved in DMF (1 mL) under an argon atmosphere. The mixture is then heated to 85° C. and stirred for 2 hours. The reaction mixture is then allowed to reach room temperature, and transferred to a separating funnel for washing with DCM and brine. The organic phase is then dried using MgSO₄ and the solvent evaporated under reduced pressure. The final product is finally purified by silica chromatography (eluent: petroleum ether:dichloromethane 1:1) to give 35.3 mg (66%) of compound 7 as an orange solid. ¹H NMR (400 MHZ, THF): δ=7.91 (d, J=8.8 Hz, 2H), 7.43 (s, 1H), 7.34 (d, J=5.0 Hz, 1H), 7.91 (d, J=8.8 Hz, 1H), 7.25 (d, J=3.6 Hz, 2H), 7.24 (s, 1H), 7.19 (t, J=3.3 Hz, 2H), 7.16 (t, J=8.3 Hz, 1H), 7.11 (q, J=3.9 Hz, 2H), 7.07 (d, J=8.9 Hz, 2H), 7.06 (s, 1H), 7.03 (dd, J=5.0, 3.7 Hz, 1H), 6.99 (dd, J=5.0, 3.7 Hz, 1H), 6.88 (s, 1H), 6.42 (d, J=8.3 Hz, 1H), 6.32 (d, J=8.4 Hz, 1H), 4.94-4.88 (m, 2H), 4.37-4.28 (m, 2H), 3.54 (s, 3H), 3.38-3.30 (m, 1H), 3.21-3.09 (m, 1H), 2.06 (s, 3H), 1.95-1.86 (m, 2H), 1.65-1.55 (m, 2H), 1.55-1.44 (m, 7H), 1.33-1.21 (m, 1H), 1.20-1.07 (m, 1H), 0.81-0.71 (m, 1H), 0.69-0.59 (m, 1H).

Method B, PTS quaterthiophene with H instead of OMe, Compound 8: A mixture containing the PTS unit diiodate (compound 4) (20 mg, 0.088 mmol), trimethylstannane 2-thiophene 11 (28 mg, 0.12 mmol), the Pd(PPh₃)₄ (5.3 mg, 0.05 mmol) is dissolved in DMF (1 mL) under an argon atmosphere. The mixture is stirred and heated to 85° C. for 15 hours, then allowed to reach room temperature. The reaction is then diluted by the addition of ethyl acetate, and the mixture transferred to a separating funnel to be washed with brine. The organic phase is then dried using MgSO₄ and the organic solvent evaporated under reduced pressure. The final product is finally purified by silica chromatography (eluent: petroleum ether:dichloromethane 1:1) to give 17.4 mg (97%) of compound 8 as an orange solid. ¹H NMR (400 MHZ, CDCl₃): δ=7.94 (d, J=8.9 Hz, 2H), 7.43 (s, 1H), 7.27 (t, J=5.2 Hz, 1H), 7.23-7.17 (m, 4H), 7.09 (d, J=3.7 Hz, 1H), 7.08 (s, 1H), 7.07 (s, 1H), 7.05 (d, J=4.3 Hz, 1H), 7.04 (s, 1H), 7.02 (dd, J=5.2, 1.6 Hz, 1H), 7.01 (s, 1H), 6.99 (dd, J=7.5, 1.7 Hz, 1H), 6.76 (t, J=7.4 Hz, 1H), 6.62 (d, J=8.2 Hz, 1H), 5.02-4.89 (m, 2H), 4.35 (t, J=5.4, 2H), 3.45-3.36 (m, 1H), 3.21-3.11 (m, 1H), 2.04 (s, 3H), 1.99-1.91 (m, 2H), 1.57-1.52 (m, 2H), 1.46 (s, 7H), 1.39-1.29 (m, 1H), 1.26 48-1.13 (m, 1H), 0.89-0.74 (m, 1H), 0.72-0.58 (m, 1H).

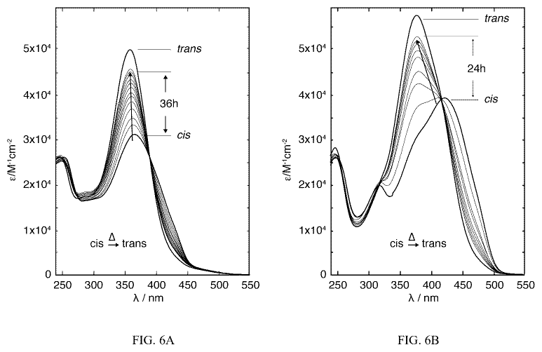

### Determination of the Conductive-to-Non-Conductive Conversion Process (Thermal Relaxation) Using UV-Vis, FIGS. 6A-6B.

The conversion times of the PTS molecules from the conductive to the non-conductive form are tested in a first step by means of UV-vis spectroscopy. PTS molecules are dissolved in an organic solvent, e.g. tetrahydrofuran (THF), to give concentrations such that the signal intensity of the absorption spectrum measured by the UV-vis spectrophotometer does not exceed the value of 0.1 in arbitrary units (au). The cuvette containing the above solution is irradiated with a wavelength of about 360 nm for 10-30 seconds, or until no further change in the absorption spectrum is detected thus indicating complete conversion from the non-conductive to the conductive form of the PTS molecule. Once complete conversion to the conductive state has been verified, the RFID is exposed to a temperature of 25° C. inside an incubator and its progress from conductive-to-non-conductive (thermal relaxation) is measured at regular intervals, e.g. every 6 hours, FIGS. 6A-6B.

### General Procedure for Converting a Commercial RFID to Integrity Sensor.

On a passive RFID, e.g. operating at 13.56 MHz (NFC tag), with a dry aluminum circuitry inlaid (dry inlay chips) on a PET (polyethylene terephthalate) support, without glue on the support and without a protective layer on the circuitry, with NXP recognition chip Ntag203 is deposited with the drop casting method a solution of about 5 mg/mL in tetrahydrofuran (THF) containing a PTS compound. The RFID is subsequently thermally treated (curing) by placing the latter on a heating plate at 40° for 5-10 min so as to facilitate the evaporation of the solvent and obtain films of PTS material. The RFID fully or partially coated by the PTS materials is then irradiated with a wavelength of about 360 nm for 5-10 minutes, or until no further radio signal change is detected thus indicating complete conversion of the PTS molecule from the non-conductive to the conductive one (activation of materials).

### Determination of the Conductive-to-Non-Conductive Process (Thermal Relaxation) Via Conversion, Reflection Confidance (S11) and Frequency at Different Temperatures, FIGS. 9A-9B.

The RFID antenna signals, before and after the deposition of the PTS materials, and after the activation of the materials, are detected by means of a network analyzer (3 GHz Nanovna V2 Vector Network Analyzer SAA2) initially at room temperature. The measured variations of the radio signal, e.g. reflection confidance (S11), frequency, and impedance (Z), establish the RFID radio signal when the PTS material is in its non-conductive state and in its conductive state.

Once the RFID signals have been detected at room temperature, the activated tag is stored in a refrigerator at 0° C., and its storage status is measured every 24 hours for 4 days. Once the retention of the activated state has been verified, the RFID is exposed to the temperature of 25° C. inside an incubator and its progress from-conductive-to-non-conductive (thermal relaxation) is measured at regular intervals, e.g. every 24 hours, FIGS. 9A-9B. As shown in FIGS. 9A-9B, the transition from conductive-to-non-conductive is a function of the time and temperature to which the RFID is exposed. In this way it is possible to measure both the intermediate signals related to the partial transition from CIS (planar π-conjugate/conductive conformation of PTS compounds) to TRANS (distorted π-conjugate/non-conductive conformation), and the final signal, once reached the complete conversion to TRANS (distorted/non-conductive π-conjugate conformation). Furthermore, it is possible to test the minimum time necessary for the total recovery of the initial (pre-activation with light) TRANS conformation (distorted π-conjugate/non-conductive conformation).

The present invention has been described with reference to preferred embodiments. It should be understood that various modifications may be made without departing from the spirit and scope of the invention. Accordingly, other embodiments are within the scope of the following claims.

What is claimed is:

1. A temperature sensor comprising a radio frequency identification (RFID), wherein the RFID comprises:
a support, wherein a heat-responsive compound characterized by a π-conjugated system is applied on the support, a conformational variation of the support varies a conductivity of the heat-responsive compound, the heat-responsive compound is more conductive in response to an absorption of light having a wavelength of 360 nm with respect to a less conductive state of the heat-responsive compound in response to an absorption of a lower wavelength light,
wherein the heat-responsive compound is applied in a most conductive conformation of the heat-responsive compound at a temperature below 0° C. and passes spontaneously, irreversibly, and unidirectionally from the most conductive conformation to a least conductive conformation as the temperature rises from 0° C. to room temperature.

2. The temperature sensor according to claim 1, wherein the heat-responsive compound has a general formula (II): 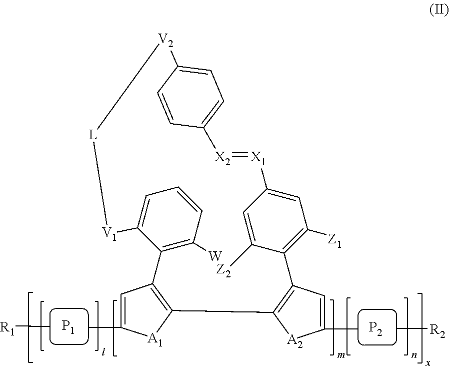
wherein:
L is a hydrocarbon chain with 5-15 carbon atoms, wherein up to 3 hydrocarbon moieties (—CH₂—) is replaced by one of the following moieties: O, NR_{L,1}, S, and/or wherein up to 3 double or triple bonds are provided, wherein pairs of a type —CH₂—CH₂— are replaced by —R_{L,2}C=CR_{L,3}—, or —N═CR_{L,4}—, with residues R_{L,1}—R_{L,4} independently selected from the group consisting of H, methyl, ethyl, propyl, isopropyl, and benzyl;
W is selected from the group consisting of H, halogen, SR_{W,1}, methyl, ethyl, OR_{W,2}, and COOR_{W,3}, with residues R_{W,1}—R_{W,3} independently selected from each other, from the group consisting of H, methyl, ethyl, propyl, isopropyl, butyl, pentanyl, hexanyl, heptanyl, octanyl, phenyl, and benzyl;
V₁ and V₂ are selected independently of each other from the following groups: CH₂, S, O, NH, COO, CO, and CONR_{V,1}, with a residue R_{V,1} selected from the group consisting of H, methyl, ethyl, propyl, isopropyl, phenyl, and benzyl;
A₁ and A₂ are selected, independently of each other, from the following groups: S, O, NH, NR_{A,1}, BH, BR_{A,1}, PR_{A,1}, PR_{A,1}R_{A,2}, Se, CH═CH, CH═N, CH—PR_{A,1}R_{A,2}, CH₂, C═O, C═CH₂, and C=CR_{A,1}R_{A,2}, with residues R_{A,1} and R_{A,2} selected, independently of each other, from the group consisting of H, methyl, ethyl, propyl, and benzyl;
X₁ and X₂ are selected, independently of each other, from the following groups: N and CH;
Z₁ and Z₂ are selected independently of each other from the following groups: halogen, methyl, ethyl, propyl, isopropyl, phenyl, benzyl, SR_{Z,1}, OR_{Z,2}, COOR_{Z,3}, NR_{Z,4}R_{Z,5}, NO₂, —CN, and —SO₃H, with residues R_{Z,1}—R_{Z,5} independently selected from the group consisting of H, methyl, ethyl, propyl, isopropyl, benzyl, primary, secondary and tertiary amines, and both alkyl and phenyl;
P₁ and P₂ are one or a mixture of π-conjugated aromatic units, wherein the P₁ and the P₂ are selected, independently of each other, from thiophene and its-derivatives of the thiophene, ethylenedioxythiophene, indacenodithiophene, cyclopentadithiophene, phenyl and derivatives of the phenyl, phenylvinylene, fluorene, indenofluorene, pyrene, pyrrole and derivatives of the pyrrole, pyrrol-2-one, maleimide, 2,1,3-benzothiadiazole, dithienylpyrrole, azole, diazole, triazole, tetrazole, indole, carbazole, or groups indicated in scheme (I) below, wherein the groups in turn are substituted or unsubstituted, and in case of substitution, R₃ is selected from hydrogen, halogen, methyl, ethyl, propyl, isopropyl, benzyl, phenyl, methoxy, ethoxy, propoxy, butoxy, pentoxy, hexaneoxy, heptanoxy, octanoxy, 3-methylheptane, 3-methoxyheptane, 7-methylpentadecane, and 7-methoxypentadecane; 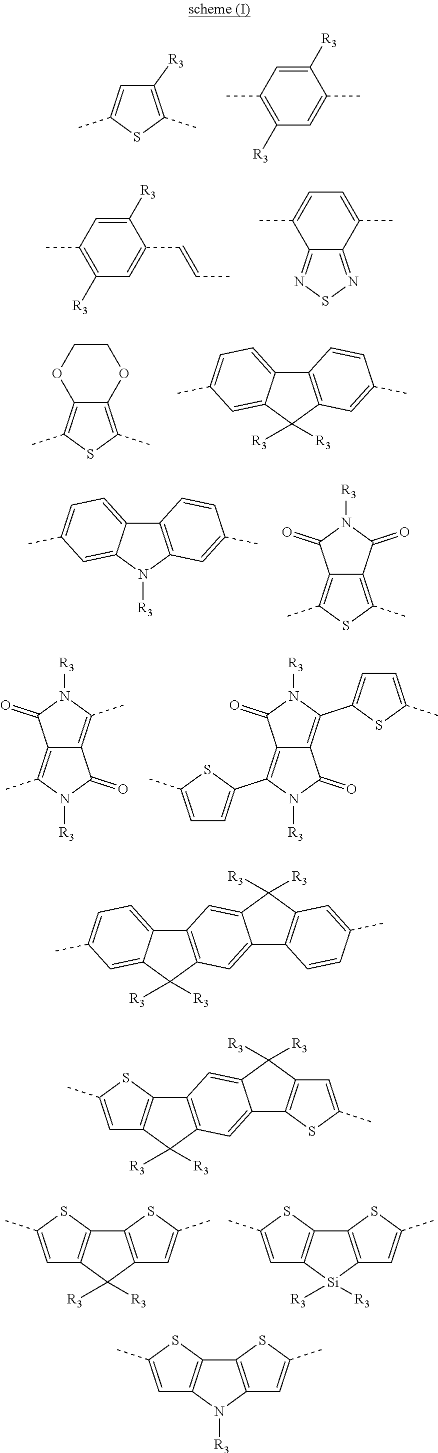
with R₁ and R₂ selected independently of each other from the following groups: H, halogen, methyl, Sn(R_{R,1})₃, B(OH)₂, OR_{R,2}, NR_{R,3}R_{R,4}, NO₂, COOR_{R,5}, COR_{R,6}, S_{R,7}, —CN, —CCR_{R,8}, —SO₃H, —CR_{R,9}=C(CN)₂, CR_{R,10}=C(CN)(COOR_{R,11}), C(CN)=C(CN)₂, methyldicyanovinyl, substituted N-ethylrhodanine and derivatives of the substituted N-ethylrhodanin, substituted and unsubstituted 3-(dicyanomethylidene) indan-1-one and derivatives of the substituted and unsubstituted 3-(dicyanomethylidene) indan-1-one, substituted and unsubstituted ferrocene and derivatives of the substituted and unsubstituted ferrocene, substituted and unsubstituted pyridine and derivatives of the substituted and unsubstituted pyridine, comprising pyridine groups, pentafluorophenyl, substituted and unsubstituted fullerene and derivatives of the substituted and unsubstituted fullerene, both alkyl and phenyl, primary, secondary and tertiary amines; with residues R_{R,1}—R_{R,11} independently selected from the group consisting of methyl, ethyl, propyl, isopropyl, phenyl, benzyl, primary, secondary and tertiary amines, and both alkyl and phenyl;
and wherein:
is selected to be an integer between 0 and 10,
m is selected to be an integer between 1 and 10,
n is selected to be an integer between 0 and 10,
x is selected as an integer between 1 and 10,000;
with a proviso that 1+n is at least 1.

3. The temperature sensor according to claim 1, wherein the heat-responsive compound is selected from compounds of formula (III), (IV), (V), (VI), and (VII):

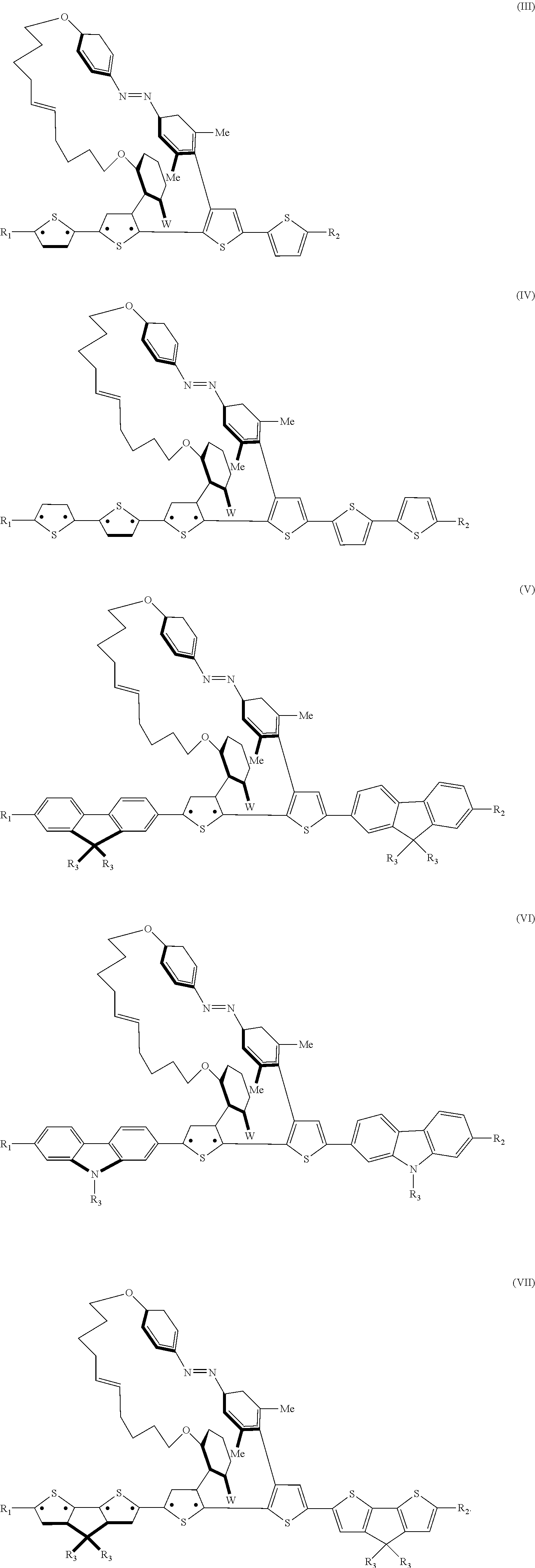

4. The temperature sensor according to claim 1, wherein the RFID further comprises:
an antenna,
optionally a microchip,
a layer of an active material comprising the heat-responsive compound, and
an insulating layer.

5. The temperature sensor according to claim 4, wherein the heat-responsive compounds is applied on the antenna.

6. The temperature sensor according to claim 4, wherein the layer of the active material is deposited on a whole antenna or on a portion of the antenna or on a portion of the support interposed between the microchip and the antenna, to join the microchip to the antenna.

7. The temperature sensor according to claim 1, wherein the RFID is of a passive type or of an active type.

8. An assembly, wherein the assembly is a temperature detector and comprises the temperature sensor according to claim 1 and an RFID reader, wherein the RFID reader is a smartphone.

9. An article comprising the temperature sensor according to claim 1 or associated with an assembly, wherein the assembly is a temperature detector and comprises the temperature sensor and an RFID reader, wherein the RFID reader is a smartphone.

10. The article according to claim 9, wherein the article is or contains products sensitive to temperature variations.

11. A composition comprising the heat-responsive compound of the temperature sensor according to claim 1 and a component selected from a carrier, an additive, and a mixture of the carrier and the additive.

12. A heterojunction formed by the composition according to claim 11 and the RFID.

13. A compound comprising a structure shown in formula 6, formula 7, formula 8, formula 19, formula 20, formula 21, formula 22, formula 23, formula 24, formula 25, formula 26, formula 27, formula 28, formula 29, or formula 30:

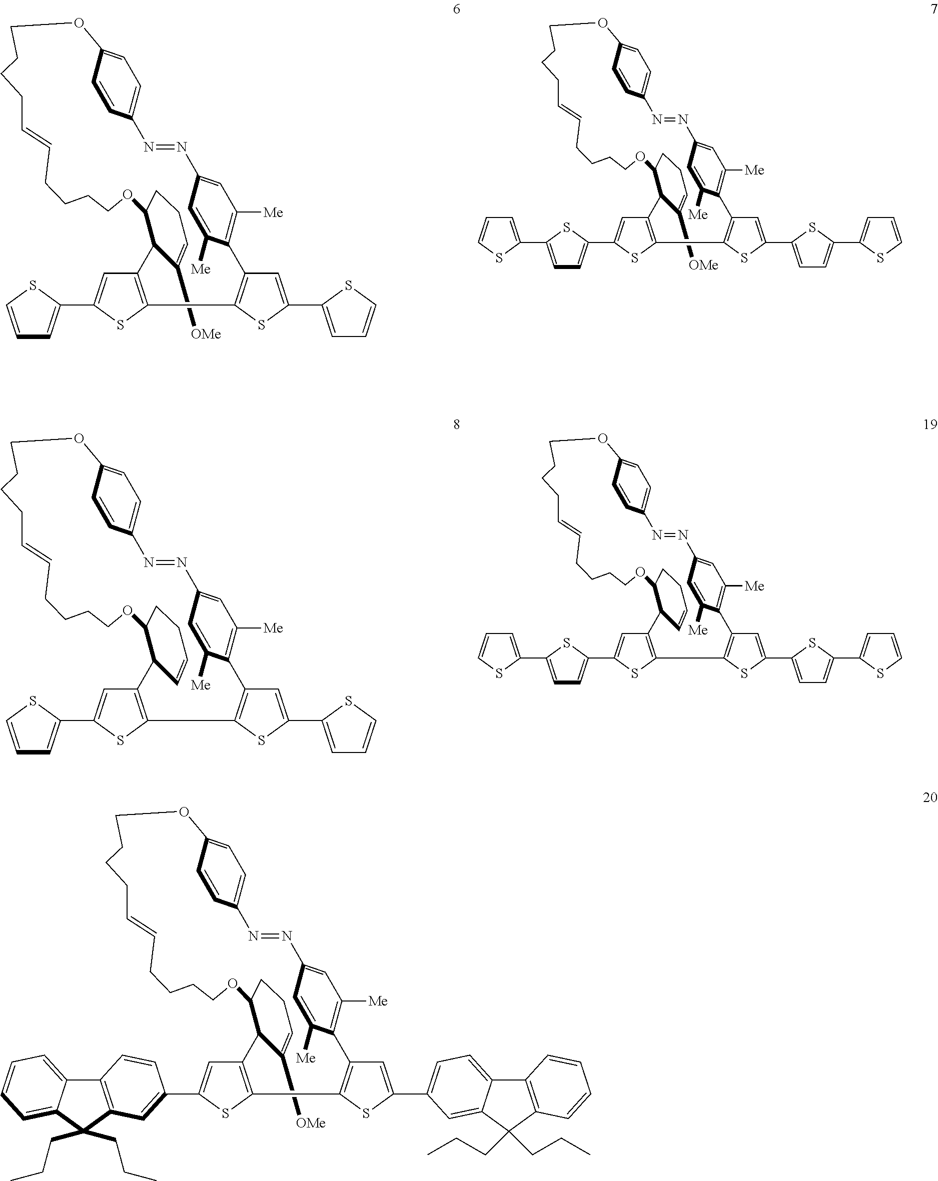

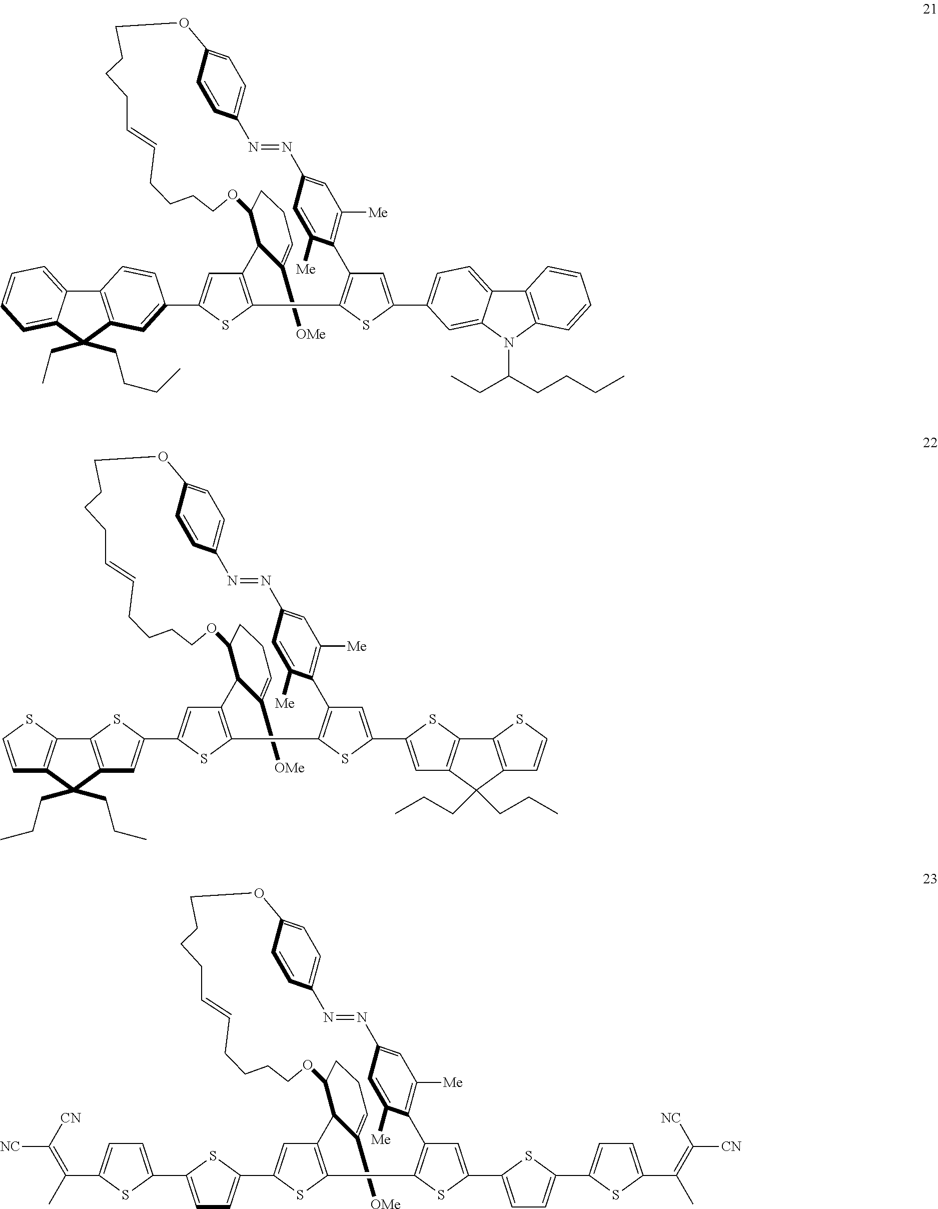

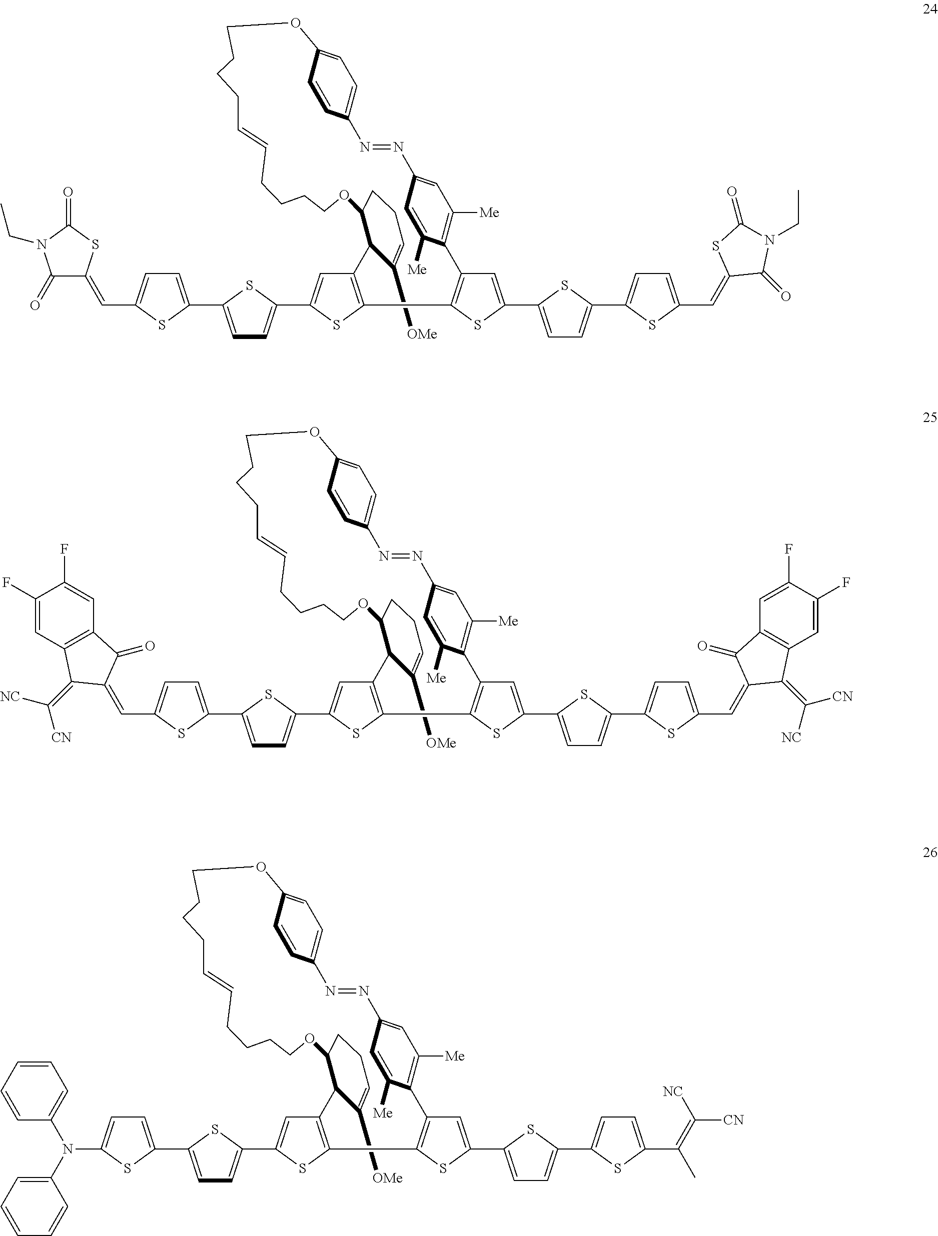

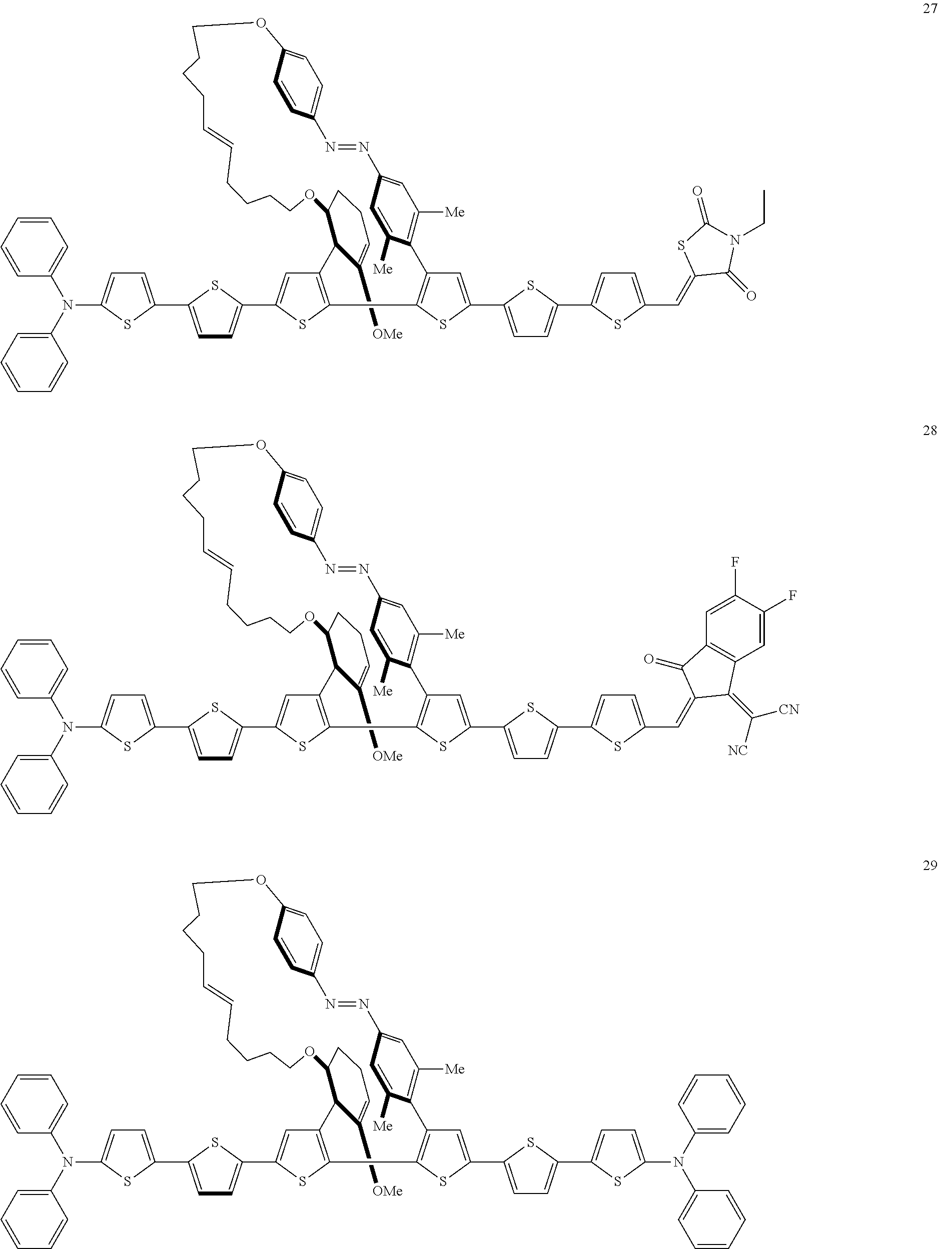

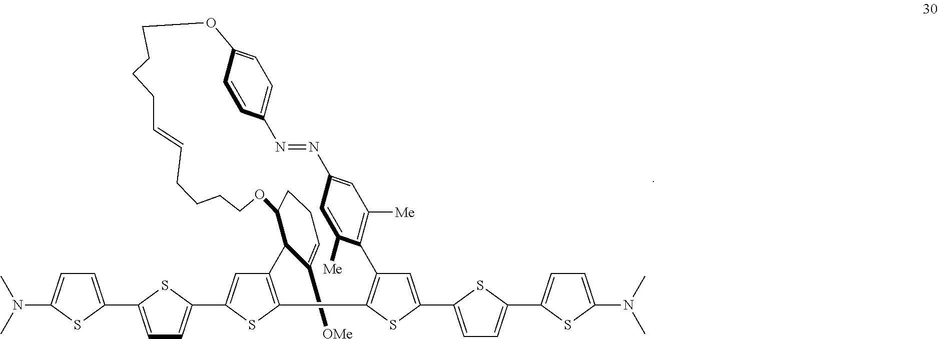

14. A method for checking a temperature continuity, comprising using the heat-responsive compound of the temperature sensor according to claim 1, or a composition in an RFID device or in a component of the RFID device or in an assembly comprising the RFID device, wherein the composition comprises the heat-responsive compound and a component selected from a carrier, an additive, and a mixture of the carrier and the additive.

15. A method for measuring a temperature continuity of a product, comprising irradiating the temperature sensor according to claim 1 by applying a radiation of a wavelength of 330-380 nm.

16. A computer-implemented method for measuring a temperature continuity of a product, comprising the following steps:
i) an acquisition of operational values of a temperature continuity detector device;
ii) an acquisition of control and threshold values of the product under an examination;
iii) an acquisition of a signal of the temperature continuity detector device;
iv) an evaluation of a progress level of a thermal relaxation;
v) counting of measurements and history;
vi) an assignment of a level of healthiness of the product.

17. A computer program comprising a code configured to perform the steps i) to vi) of the computer-implemented method according to claim 16 when executed on a computer.

18. The computer program according to claim 17, wherein the computer program is executed by means of a smartphone application or other RFID reader.

19. A computer medium comprising the computer program according to claim 17.

20. A device or computer or computing medium implementing the computer-implemented method according to claim 16.

21. The device or computer or computing medium according to claim 20, wherein the device or computer or computing medium is selected from: an RFID reader or a mobile device comprising a scanner, a mobile phone, a personal digital assistant (PDA), a smartphone, a tablet, or a laptop.
